(19) United States
(12) Patent Application Publication
(10) Pub. No.: US 2013/0156863 A1
(43) Pub. Date: Jun. 20, 2013

(54) METHODS OF PREPARING CHORION TISSUE AND PRODUCTS DERIVED THEREFROM

(71) Applicants: Scheffer Tseng, Pinecrest, FL (US); Ek Kia Tan, Miami, FL (US); Lorraine Siok May Chua, Miami, FL (US)

(72) Inventors: Scheffer Tseng, Pinecrest, FL (US); Ek Kia Tan, Miami, FL (US); Lorraine Siok May Chua, Miami, FL (US)

(73) Assignee: TISSUETECH, INC., Miama, FL (US)

(21) Appl. No.: 13/704,231

(22) PCT Filed: Jun. 30, 2011
(86) PCT No.: PCT/US11/42679

§ 371 Date: Feb. 26, 2013

Related U.S. Application Data
(60) Provisional application No. 61/360,263, filed on Jun. 30, 2010.

(51) Int. Cl. A61K 35/50

(52) U.S. Cl. CPC A61K 35/50
USPC 424/583; 435/317.1

(57) ABSTRACT

Disclosed herein, in certain instances, are tissue grafts derived from chorion or amnio-chorion. Further disclosed herein, in certain instances, are use for tissue grafts derived from chorion or amnio-chorion.

### RELATED APPLICATIONS

This application is the National Stage under 35 USC §371 of International Application No. PCT/US2011/042679 filed Jun. 30, 2011, which claims priority to U.S. provisional patent application 61/360,263 filed Jun. 30, 2010, which is incorporated in its entirety by reference.

### STATEMENT AS TO FEDERALLY SPONSORED RESEARCH

The invention was made with United States government support under grant number 2R44EY017497-02A1 awarded by the National Institutes of Health. The United States government may have certain rights in the invention.

### BACKGROUND OF THE INVENTION

The chorion consists of two layers: an outer formed by the trophoblast, and an inner formed by the somatic mesoderm; amnion contacts the latter. The trophoblast is made up of an internal layer of cubical or prismatic cells, the cytotrophoblast or layer of Langhans, and an external layer of richly nucleated protoplasm devoid of cell boundaries, the syncytiotrophoblast.

### SUMMARY OF THE INVENTION

Disclosed herein, in certain embodiments, is a tissue product, comprising: isolated chorion that is substantially free of cells with metabolic activity, wherein the natural biological activity of the isolated chorion is substantially maintained. In some embodiments, the natural biological activity is the activity of polypeptides, polysaccharides, and lipids found in the chorion. In some embodiments, the natural biological activity is the activity of TSG-6, HCHA, PTX3, and HC1. In some embodiments, the natural biological activity of the isolated chorion has only decreased by about 1% as compared to the natural biological activity of fresh chorion. In some embodiments, the natural biological activity of the isolated chorion has only decreased by about 5% as compared to the natural biological activity of fresh chorion. In some embodiments, the natural biological activity of the isolated chorion has only decreased by about 10% as compared to the natural biological activity of fresh chorion. In some embodiments, the natural structural integrity of the isolated chorion is substantially maintained. In some embodiments, the natural structural integrity of the stroma matrix and basement membrane are substantially maintained. In some embodiments, the natural structural integrity of the isolated chorion has only decreased by about 1% as compared to the natural structural integrity of fresh chorion. In some embodiments, the natural structural integrity of the isolated chorion has only decreased by about 5% as compared to the natural structural integrity of fresh chorion. In some embodiments, the natural structural integrity of the isolated chorion has only decreased by about 10% as compared to the natural structural integrity of fresh chorion. In some embodiments, the tissue product is pain-relieving, anti-inflammatory, anti-scarring, anti-angiogenic, anti-adhesive, or promotes wound healing when contacted with host tissue. In some embodiments, substantially all red blood cells have been removed. In some embodiments, the tissue product is cryopreserved, lyophilized, terminally sterilized, pulverized, homogenized, or any combination thereof. In some embodiments, the tissue product is centrifuged to generate a chorion extract. In some embodiments, the tissue product is substantially-flattened sheet. In some embodiments, the tissue product is a pulverized or homogenized. In some embodiments, the tissue product is substantially free of HIV-1, HIV-2, HTLV-1, hepatitis B and C, West Nile virus, cytomegalovirus, human transmissible spongiform encephalopathy, and treponema pallidum.

Disclosed herein, in certain embodiments, is a method of producing a tissue product, comprising: (a) isolating chorion from placenta, to generate isolated chorion; and (b) inhibiting the metabolic activity of substantially all cells of the isolated chorion; wherein the method of producing substantially maintains the natural biological activity of the isolated chorion. In some embodiments, the method of producing substantially maintains the natural biological activity of polypeptides, polysaccharides, and lipids found in the chorion. In some embodiments, the method of producing substantially maintains the natural biological activity of TSG-6, HCHA, PTX3, and HC1. In some embodiments, the natural biological activity of the isolated chorion has only decreased by about 1% as compared to the natural biological activity of fresh chorion. In some embodiments, the natural biological activity of the isolated chorion has only decreased by about 5% as compared to the natural biological activity of fresh chorion. In some embodiments, the natural biological activity of the isolated chorion has only decreased by about 10% as compared to the natural biological activity of fresh chorion. In some embodiments, the method of producing substantially maintains the natural structural integrity of the isolated chorion. In some embodiments, the method of producing substantially maintains the natural structural integrity of the stroma matrix and basement membrane. In some embodiments, the natural structural integrity of the isolated chorion has only decreased by about 1% as compared to the natural structural integrity of fresh chorion. In some embodiments, the natural structural integrity of the isolated chorion has only decreased by about 5% as compared to natural structural integrity of fresh chorion. In some embodiments, the natural structural integrity of the isolated chorion has only decreased by about 10% as compared to the natural structural integrity of fresh chorion. In some embodiments, the placenta is obtained from a human, non-primate human, cow or pig. In some embodiments, the tissue product is pain-relieving, anti-inflammatory, anti-scarring, anti-angiogenic, anti-adhesive, or promotes wound healing when contacted with host tissue. In some embodiments, the method further comprises inhibiting the metabolic activity of substantially all cells found on the tissue product by freezing or lyophilizing the placenta or isolated chorion. In some embodiments, the method further comprises removing substantially all red blood cells. In some embodiments, the method further comprises lyophilizing, cryopreserving, pulverizing, homogenizing, or terminally sterilizing the tissue product. In some embodiments, the method further comprises centrifuging the tissue product to generate a chorion extract. In some embodiments, the method further comprises screening the placenta or chorion for HIV-1, HIV-2, HTLV-1, hepatitis B and C, West Nile virus, cytomegalovirus, human transmissible spongiform encephalopathy, and treponema pallidum.

Disclosed herein, in certain embodiments, is a tissue product, comprising: isolated amnion-chorion that is substantially free of cells with metabolic activity, wherein the natural biological activity of the isolated amnion-chorion is substantially maintained. In some embodiments, the natural biological activity is the activity of polypeptides, polysaccharides, and lipids found in the amnion-chorion. In some embodiments, the natural biological activity is the activity of TSG-6, HCHA, PTX3, and HC1. In some embodiments, the natural biological activity of the isolated amnion-chorion has only decreased by about 1% as compared to the natural biological activity of fresh amnion-chorion. In some embodiments, the natural biological activity of the isolated amnion-chorion has only decreased by about 5% as compared to the natural biological activity of fresh amnion-chorion. In some embodiments, the natural biological activity of the isolated amnion-chorion has only decreased by about 10% as compared to the natural biological activity of fresh amnion-chorion. In some embodiments, the natural structural integrity of the isolated n-chorion is substantially maintained. In some embodiments, the natural structural integrity of the stroma matrix and basement membrane are substantially maintained. In some embodiments, the natural structural integrity of the isolated amnion-chorion has only decreased by about 1% as compared to the natural structural integrity of fresh amnion-chorion. In some embodiments, the natural structural integrity of the isolated amnion-chorion has only decreased by about 5% as compared to the natural structural integrity of fresh amnion-chorion. In some embodiments, the natural structural integrity of the isolated amnion-chorion has only decreased by about 10% as compared to the natural structural integrity of fresh amnion-chorion. In some embodiments, the tissue product is pain-relieving, anti-inflammatory, anti-scarring, anti-angiogenic, anti-adhesive, or promotes wound healing when contacted with host tissue. In some embodiments, substantially all red blood cells have been removed. In some embodiments, the tissue product is cryopreserved, lyophilized, terminally sterilized, pulverized, homogenized, or any combination thereof. In some embodiments, the tissue product is centrifuged to generate an amnion-chorion extract. In some embodiments, the tissue product is substantially-flattened sheet. In some embodiments, the tissue product is pulverized or homogenized. In some embodiments, the tissue product is substantially free of HIV-1, HIV-2, HTLV-1, hepatitis B and C, West Nile virus, cytomegalovirus, human transmissible spongiform encephalopathy, and treponema pallidum.

Disclosed herein, in certain embodiments, is a method of producing a tissue product, comprising: (a) isolating amnion-chorion from placenta, to generate isolated amnion-chorion; and (b) inhibiting the metabolic activity of substantially all cells of the isolated amnion-chorion; wherein the method of producing substantially maintains the natural biological activity of the isolated amnion-chorion. In some embodiments, the method of producing substantially maintains the natural biological activity of polypeptides, polysaccharides, and lipids found in the amnion-chorion. In some embodiments, the method of producing substantially maintains the natural biological activity of TSG-6, HCHA, PTX3, and HC1. In some embodiments, the natural biological activity of the isolated amnion-chorion has only decreased by about 1% as compared to the natural biological activity of fresh amnion-chorion. In some embodiments, the natural biological activity of the isolated amnion-chorion has only decreased by about 5% as compared to the natural biological activity of fresh amnion-chorion. In some embodiments, the natural biological activity of the isolated amnion-chorion has only decreased by about 10% as compared to the natural biological activity of fresh amnion-chorion. In some embodiments, the method of producing substantially maintains the natural structural integrity of the isolated amnion-chorion. In some embodiments, the method of producing substantially maintains the natural structural integrity of the stroma matrix and basement membrane. In some embodiments, the natural structural integrity of the isolated amnion-chorion has only decreased by about 1% as compared to the natural structural integrity of fresh amnion-chorion. In some embodiments, the natural structural integrity of the isolated amnion-chorion has only decreased by about 5% as compared to natural structural integrity of fresh amnion-chorion. In some embodiments, the natural structural integrity of the isolated amnion-chorion has only decreased by about 10% as compared to the natural structural integrity of fresh amnion-chorion. In some embodiments, the placenta is obtained from a human, non-primate human, cow or pig. In some embodiments, the tissue product is pain-relieving, anti-inflammatory, anti-scarring, anti-angiogenic, anti-adhesive, or promotes wound healing when contacted with host tissue. In some embodiments, the method further comprises inhibiting the metabolic activity of substantially all cells found on the tissue product by freezing or lyophilizing the placenta or isolated amnion-chorion. In some embodiments, the method further comprises removing substantially all red blood cells. In some embodiments, the method further comprises lyophilizing, cryopreserving, pulverizing, homogenizing, or terminally sterilizing the tissue product. In some embodiments, the method further comprises centrifuging the tissue product to generate an amnion-chorion extract. In some embodiments, the method further comprises screening the placenta or chorion for HIV-1, HIV-2, HTLV-1, hepatitis B and C, West Nile virus, cytomegalovirus, human transmissible spongiform encephalopathy, and treponema pallidum.

Disclosed herein, in certain embodiments, is the use of a tissue product described herein to reduce pain, promote wound healing and reduce inflammation, scarring, angiogenesis, and adhesive in a plurality of cells.

Disclosed herein, in certain embodiments, is the use of a tissue product described herein as a wound covering for an injured tissue.

Disclosed herein, in certain embodiments, is the use of a tissue product described herein for repairing, supplementing, or augmenting damaged tissue.

Disclosed herein, in certain embodiments, is the use of a tissue product described herein as an anti-adhesive barrier.

### DESCRIPTION OF FIGURES

FIG. 1 illustrates that purified chorion extract results in a higher release of PTX3 and HC1 as compared to purified amniotic membrane extract.

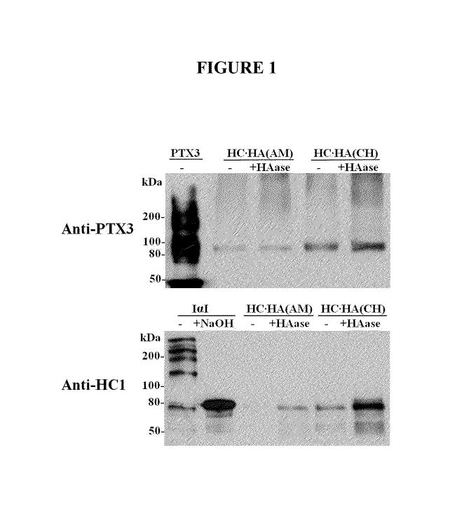

FIG. 2 illustrates that purified chorion extract is 25 fold more potent than that from amniotic membrane extract regarding HUVEC proliferation inhibition.

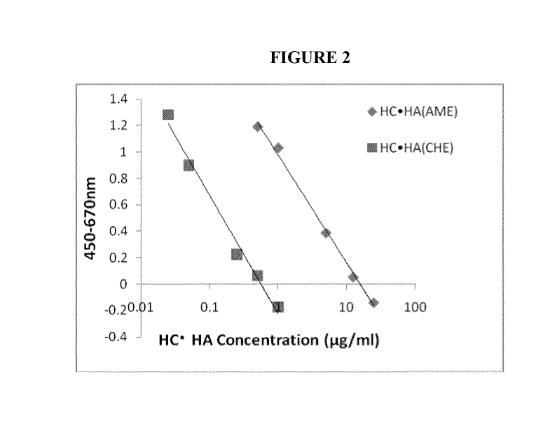

FIG. 3 illustrates wound healing using a tissue product comprising isolated chorion for treatment of a Venous Ulcer.

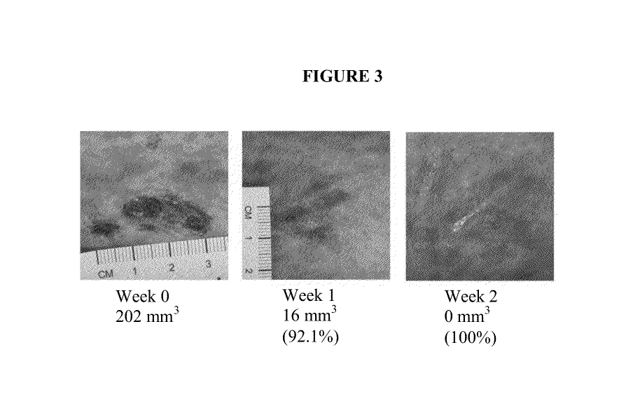

FIG. 4 illustrates wound healing using a tissue product comprising isolated chorion for treatment of a Venous Ulcer.

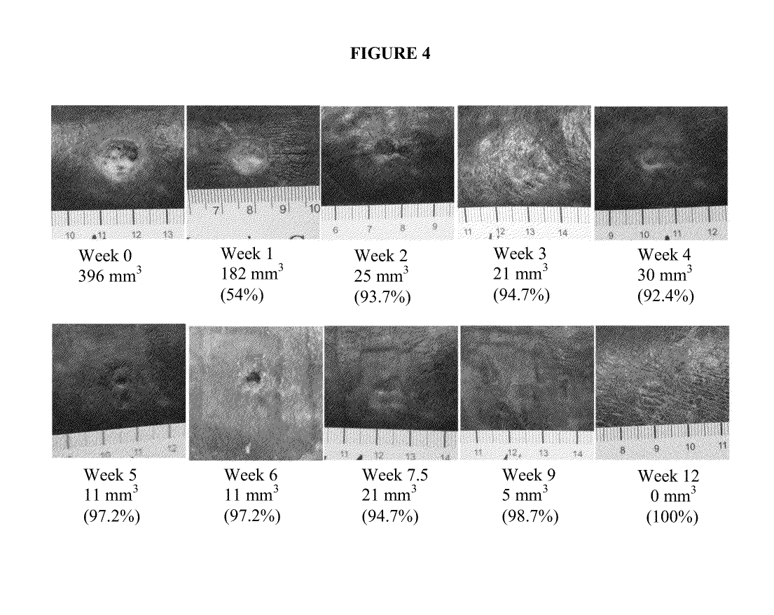

FIG. 5 illustrates wound healing using a tissue product comprising isolated chorion for treatment of an Arterial Insufficiency Ulcer.

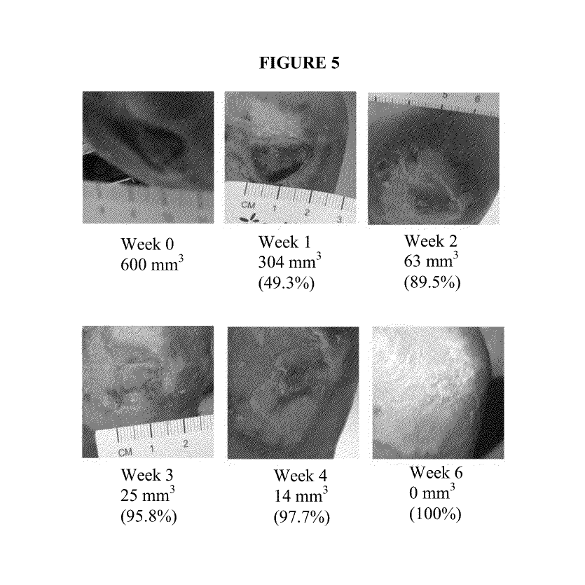

FIG. 6 illustrates wound healing using a tissue product comprising isolated chorion for treatment of an Arterial Insufficiency Ulcer.

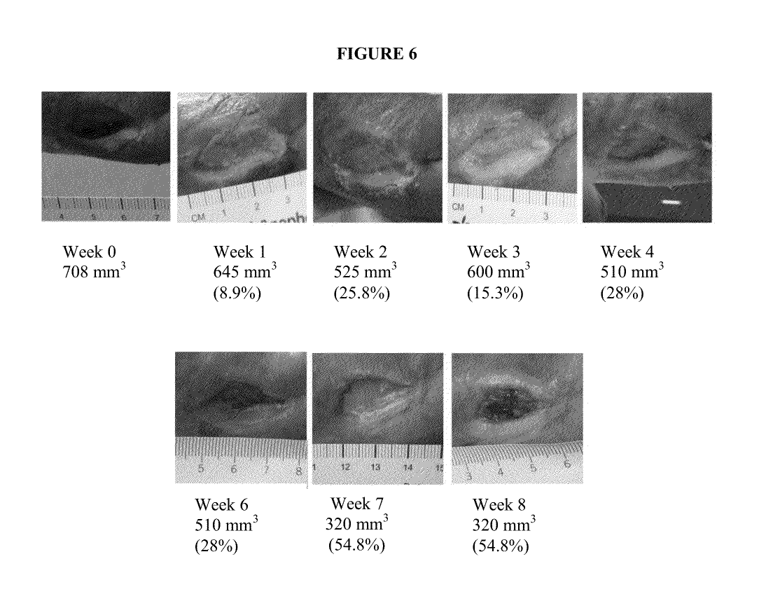

### DETAILED DESCRIPTION OF THE INVENTION

There is a need for a product derived from a natural source (e.g., human, non-human primate, pig or cow) that repairs, reconstructs, replaces or supplements tissue (e.g., tendons or nerves) that has been damaged, compromised, or is even missing. In order to fulfill the aforementioned needs such a product should be regenerative, anti-inflammatory, anti-scarring, anti-angiogenic, anti-adhesive, promote wound healing, durable, strong, flexible, and conformable. Such a product should also preferably serve as a niche for the in vivo growth and differentiation of stem cells.

Disclosed herein, in certain embodiments, are tissue products comprising chorion, amnion-chorion, or a combination thereof. The tissue products are derived from a natural source (e.g., human, non-human primate, cow or pig) and are pain-relieving, regenerative, anti-inflammatory, anti-scarring, anti-angiogenic, anti-adhesive, durable, strong, flexible, and conformable. In some embodiments, the tissue products are used as wound coverings. In some embodiments, the tissue products are used as a patch over a device. In some embodiments, the tissue products are used as an anti-adhesive barrier. In some embodiments, the tissue products are used for wound repair. In some embodiments, the tissue products are in the form of a substantially-flat sheet. In some embodiments, the tissue products are pulverized or homogenized.

### CERTAIN DEFINITIONS

As used herein, “human cells, tissues, or cellular or tissue-based products (HCT/Ps)” means articles containing or consisting of human cells or tissues that are intended for implantation, transplantation, infusion, or transfer into a human recipient.

As used herein, “human tissue” means any tissue derived from a human body. In some embodiments, the human tissue is chorion. In some embodiments, the human tissue is amnion-chorion.

As used herein, “minimal manipulation” means (1) for structural tissue, processing that does not alter the original relevant characteristics of the tissue relating to the tissue's utility for reconstruction, repair, or replacement; and (2) for cells or nonstructural tissues, processing that does not alter the relevant biological characteristics of cells or tissues.

As used herein, ‘homologous use” means the repair, reconstruction, replacement, or supplementation of a recipient's cells or tissues with an HCT/P that performs the same basic function or functions in the recipient as in the donor.

As used herein, “processing” means any activity performed on an HCT/P, other than recovery, donor screening, donor testing, storage, labeling, packaging, or distribution, such as testing for microorganisms, preparation, sterilization, steps to inactivate or remove adventitious agents, preservation for storage, and removal from storage.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “mono-layer” by producing glycoprotein-like material that causes the cells to adhere to the plastic or glass of the Petri dish. A layer of culture medium, containing nutrients suitable for cell growth, is then placed on top of the mono-layer, and the culture is incubated to promote the growth of the cells.

As used herein, “sheet” means any continuous expanse or surface. In some embodiments, a tissue product described herein is a flat sheet. The sheet can be any shape and size. In some embodiments, the sheet is a square, circle, triangle, or rectangle. In some embodiments, the sheet comprises multiple layers of chorion, amnion-chorion, or a combination thereof.

As used herein, the term “subject” is used to mean any animal, preferably a mammal, including a human or non-human. The terms patient, subject, and individual are used interchangeably. None of the terms are to be interpreted as requiring the supervision of a medical professional (e.g., a doctor, nurse, physician's assistant, orderly, hospice worker).

“Substantially isolated” or “isolated” when used in the context of chorion or amnion-chorion means that the chorion or amnion-chorion is separated from other non-chorion materials or amnion-chorion materials not of interest.

The terms “treat,” “treating” or “treatment,” as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

As used herein, the terms “durable” and “strong” mean that chorion or amnion-chorion is an elastic material with higher yield strength, higher stiffness, higher pull strength, higher tensile strength, and higher suture pull-out strength than placental amniotic membrane (PAM).

As used herein, the phrase “the natural biological activity is substantially maintained”, and variations thereof, means that when compared to the biological activity of fresh chorion or amnion-chorion, the biological activity of a chorion or amnion-chorion tissue product described herein (e.g., a flat tissue product sheet, a pulverized tissue product, or a homogenized tissue product) has only decreased by about 1%, about 5%, about 10%, about 15%, about 20%, about 25%, about 30%, about 35%, about 40%, about 50%, about 60%, even though the composition is substantially free of cells with metabolic activity.

As used herein, “biological activity” means the activity of biological molecules (e.g., polypeptides, polysaccharides, and lipids) found in the membrane. Examples of biologically active molecules include, but are not limited to, TSG-6, HCHA, PTX3, and HC1 (see, example 27). In some embodiments, the activity of biological molecules found in chorion and amnion-chorion (and isolated derivatives thereof) are pain-relieving, anti-inflammatory, anti-scarring, anti-angiogenic, or anti-adhesive. In some embodiments, the activity of biological molecules found in chorion and amnion-chorion (and isolated derivatives thereof) promotion of wound healing.

As used herein, the phrase “the natural structural integrity is substantially maintained”, and variations thereof, means that when compared to the structural integrity of fresh chorion or amnion-chorion, the natural structural integrity of a chorion or amnion-chorion tissue product described herein (e.g., a flat tissue product sheet, a pulverized tissue product, or a homogenized tissue product) has only decreased by about 1%, about 5%, about 10%, about 15%, about 20%, about 25%, about 30%, about 35%, about 40%, about 50%, about 60%, even though the composition is substantially free of cells with metabolic activity.

As used herein, “structural integrity” means the integrity of components of stroma matrix and basement membrane that make up fresh chorion or the amnion-chorion. In some embodiments, the structural integrity of the chorion results in suture pull out strength.

As used herein, the term “host” means a subject receiving tissue product disclosed herein (e.g., a flat tissue product sheet, a pulverized tissue product, or a homogenized tissue product). In some embodiments, the subject is a human. In some embodiments, the subject is a non-human mammal (e.g., a primate).

As used herein, the term “host tissue” means the tissue of a subject receiving tissue product disclosed herein (e.g., a flat tissue product sheet, a pulverized tissue product, or a homogenized tissue product). In some embodiments, the host tissue is living. In some embodiments, the host tissue is dead (e.g., necrotic).

### Chorion and Amnion-chorion

Chorion is an opaque membrane. The chorion has two layers: an outer formed by the trophoblast, and an inner formed by the somatic mesoderm. The amnion-chorion is a combination of the placental chorion and amnion. The avascular amnion is adherent to the inner layer of the chorion. In some embodiments, the avascular amnion is initially separated from the chorion and later combined with the chorion during processing.

The chorion and the amnion-chorion possess several regenerative properties. Both reduce inflammation, reduce angiogenesis, reduce scarring, and reduce adhesive. Further, the chorion and the amnion-chorion, when their natural biological activity and natural structural integrity is maintained, serve as a natural niche for stem cells. Thus, wounds covered with a tissue product described herein (e.g., a flat tissue product sheet, a pulverized tissue product, or a homogenized tissue product) often display an increased rate of healing as compared to wounds covered with a tissue graft made of alternative materials.

### Generation of Flat Tissue Product Sheets

Disclosed herein, in some embodiments, is a flat tissue product sheet, comprising: an isolated chorion or isolated amnion-chorion that is substantially free of cells with metabolic activity wherein the natural biological activity of the isolated chorion or isolated amnion-chorion is substantially maintained. In some embodiments, the flat tissue product sheet is substantially free of HIV-1, HIV-2, HTLV-1, hepatitis B and C, West Nile virus, cytomegalovirus, human transmissible spongiform encephalopathy (e.g., Creutzfeldt-Jakob disease) and treponema pallidum. In some embodiments, the structural integrity of the isolated chorion or isolated amnion-chorion is substantially maintained.

Further disclosed herein, in certain embodiments, a method of producing a flat tissue product sheet, comprising: obtaining placenta, and separating the chorion or amnion-chorion from the rest of the placenta, wherein the natural biological activity of the chorion or amnion-chorion is substantially maintained. In some embodiments, the natural structural integrity of the chorion or amnion-chorion is substantially maintained.

### Obtaining Placenta

Placenta is recovered from any suitable source (e.g., a hospital or tissue bank). Placenta can be obtained from any mammal, such as a human, non-human primate, cow or pig. The placenta may be frozen, previously frozen, or fresh (i.e., not frozen).

Where the placenta is not processed into a pulverized tissue product immediately after it has been obtained, it is processed for storage (e.g., it is frozen or dried). In some embodiments, the placenta is frozen for storage. In some embodiments, the placenta is frozen at −80° C. In some embodiments, the placenta is frozen until donor and specimen eligibility has been determined. In some embodiments, the placenta is placed in a cryo-preservative before being frozen.

In some embodiments, freezing the placenta kills substantially all cells found in the chorion or amnion-chorion. In some embodiments, freezing the placenta kills substantially all cells found in the chorion or amnion-chorion while maintaining or increasing the biological activity of the chorion or isolated chorion (e.g., its anti-inflammatory, anti-scarring, anti-antigenic, and anti-adhesive properties) relative to fresh (i.e., non-frozen) chorion or amnion-chorion.

In some embodiments, freezing the placenta results in the loss of metabolic activity in substantially all cells found in the chorion or amnion-chorion. In some embodiments, freezing the placenta results in the loss of metabolic activity in substantially all cells found in the chorion or amnion-chorion while maintaining or increasing the biological activity of the chorion or amnion-chorion chorion (e.g., its anti-inflammatory, anti-scarring, anti-antigenic, and anti-adhesive properties) relative to fresh (i.e., non-frozen) chorion or amnion-chorion.

In some embodiments, the placenta is dried. In some embodiments, drying the placenta kills substantially all cells found in the chorion or isolated chorion. In some embodiments, drying the placenta results in the loss of metabolic activity in substantially all cells found in the chorion or isolated chorion.

### Initial Processing of Placenta

All processing is done following Good Tissue Practices (GTP) to ensure that no contaminants are introduced into the tissue products.

The placenta is tested for HIV-1, HIV-2, HTLV-1, hepatitis B and C, West Nile virus, cytomegalovirus, human transmissible spongiform encephalopathy (e.g., Creutzfeldt-Jakob disease) and treponema pallidum using FDA licensed screening test. Any indication that the tissue is contaminated with HIV-1, HIV-2, HTLV-1, hepatitis B and C, West Nile virus, or cytomegalovirus results in the immediate quarantine and subsequent destruct of the tissue specimen.

Further, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and treponema pallidum results in the immediate quarantine and subsequent destruct of the tissue specimen.

In some embodiments, the placenta is frozen. In some embodiments, the placenta is fresh (i.e., not frozen). If the placenta is fresh (i.e., not frozen), it is processed as described below immediately.

### Further Processing

In some embodiments, the chorion or amnion-chorion is not isolated from the placenta before further processing begins. In some embodiments, the chorion or amnion-chorion is isolated from the placenta (generating isolated chorion or isolated amnion-chorion) before further processing begins,

In some embodiments, substantially all of the blood is removed from the placenta, isolated chorion, or isolated amnion-chorion. In some embodiments, some blood is removed from the placenta, chorion, or amnion-chorion. In some embodiments, the blood is not removed from the placenta, chorion, or amnion-chorion.

In some embodiments, the placenta, chorion, or amnion-chorion is washed with buffer with agitation to remove excess blood and tissue. In some embodiments, washing with agitation reduces the wash time.

In some embodiments, the placenta, chorion, or amnion-chorion is washed with an isotonic buffer or tissue culture media. In some embodiments, the placenta, chorion, or amnion-chorion is washed with saline. In some embodiments, the placenta, chorion, or amnion-chorion is washed with PBS. In some embodiments, the placenta, chorion, or amnion-chorion is washed with PBS1×. In some embodiments, the placenta, chorion, or amnion-chorion is washed with a TRIS-buffered saline. In some embodiments, the placenta, chorion, or amnion-chorion is washed with a HEPES-buffered saline. In some embodiments, the placenta, chorion, or amnion-chorion is washed with Ringer's solution. In some embodiments, the placenta, chorion, or amnion-chorion is washed with Hartmann's solution. In some embodiments, the placenta, chorion, or amnion-chorion is washed with EBSS. In some embodiments, the placenta, chorion, or amnion-chorion is washed with HBSS. In some embodiments, the placenta, chorion, or amnion-chorion is washed with Tyrode's Salt Solution. In some embodiments, the placenta, chorion, or amnion-chorion is washed with Gey's Balanced Salt Solution. In some embodiments, the placenta, chorion, or amnion-chorion is washed with DMEM. In some embodiments, the placenta, chorion, or amnion-chorion is washed with EMEM. In some embodiments, the placenta, chorion, or amnion-chorion is washed with GMEM. In some embodiments, the placenta, chorion, or amnion-chorion is washed with RPMI.

In some embodiments, the placenta, chorion, or amnion-chorion is washed with an isotonic buffer or tissue culture media, and any suitable antibiotic. In some embodiments, the antibiotic is ciprofloxacin, amphotericin B, penicillin, streptomycin, neomycin or a combination thereof. In some embodiments, the antibiotic is ciprofloxacin and amphotericin B. In some embodiments, the antibiotic is penicillin, streptomycin, neomycin, and amphotericin B.

Processing of the Chorion or Amnion-Chorion into a Flattened Tissue Product

In some embodiments, isolated chorion or isolated amnion-chorion is flattened following separation from the placenta, generating a flattened tissue product comprising isolated chorion or isolated amnion-chorion.

In some embodiments, the flattened tissue product comprising isolated chorion or isolated amnion-chorion is cut into multiple sections (e.g., using a scalpel). The size of the sections depends on the desired use of the flattened tissue product (e.g., tissue graft).

In some embodiments, multiple layers of isolated chorion and/or isolated amnion-chorion are combined to generate a layered, flattened tissue product. The layered, flattened tissue product is any suitable thickness. In some embodiments, the layered, flattened tissue product comprises two, three, four, five, six, seven, eight, nine, or ten layers of isolated chorion and/or isolated amnion-chorion. In some embodiments, the layered, flattened tissue product comprises more than ten layers of isolated chorion and/or isolated amnion-chorion.

In some embodiments, the tissue product is optionally contacted with a substrate (i.e., a supportive backing). In some embodiments, the tissue product is not contacted with a substrate. In some embodiments, the tissue product does not require a particular orientation relative to the substrate (i.e., any side of the chorion or amnion-chorion may be in contact with the substrate). In some embodiments, the tissue product is orientated such that the epithelial layer is in contact with the substrate.

Preferably, the substrate does not comprise bleach or chlorine, and is stable especially when placed in storage medium. In some embodiments, the substrate is nitrocellulose paper (NC). In some embodiments, the substrate is nylon membrane (NM). In some embodiments, the substrate is polyethersulfone membrane (PES).

In some embodiments, the side of the substrate which is not in contact with the isolated chorion or isolated amnion-chorion (i.e., the back side of the substrate) is marked. For example, the back side of the substrate is gridded or lettering is placed on the back side.

In some embodiments, the tissue product is cut into pieces following attachment to the substrate.

### Generation of Pulverized Tissue Product

Disclosed herein, in some embodiments, is a pulverized tissue product, comprising: an isolated chorion or isolated amnion-chorion that is substantially free of cells with metabolic activity, wherein the natural biological activity of the isolated chorion or isolated amnion-chorion is substantially maintained. In some embodiments, the pulverized tissue product sheet is substantially free of HIV-1, HIV-2, HTLV-1, hepatitis B and C, West Nile virus, cytomegalovirus, human transmissible spongiform encephalopathy (e.g., Creutzfeldt-Jakob disease) and treponema pallidum. In some embodiments, the structural integrity of the isolated chorion or isolated amnion-chorion is substantially maintained.

Further disclosed herein, in certain embodiments, are methods of producing a pulverized tissue product, comprising: obtaining placenta, and separating the chorion or amnion-chorion from the rest of the placenta, wherein the natural biological activity of the chorion or amnion-chorion is substantially maintained. In some embodiments, the natural structural integrity of the chorion or amnion-chorion is substantially maintained.

Placenta is recovered from any suitable source (e.g., a hospital or tissue bank). Placenta can be obtained from any mammal, such as a human, non-human primate, cow or pig. The placenta may be frozen, previously frozen, or fresh (i.e., not frozen).

Where the placenta is not processed into a pulverized tissue product immediately after it has been obtained, it is processed for storage (e.g., it is frozen or dried). In some embodiments, the placenta is frozen for storage. The placenta is frozen at any suitable temperature (e.g., −80° C.). In some embodiments, the placenta is frozen at −80° C. In some embodiments, the placenta is frozen until donor and specimen eligibility has been determined. In some embodiments, the placenta is placed in a cryo-preservative before being frozen.

In some embodiments, freezing the placenta kills substantially all cells found in the chorion or amnion-chorion. In some embodiments, freezing the placenta kills substantially all cells found in the chorion or amnion-chorion while maintaining or increasing the biological activity of the chorion or isolated chorion (e.g., its anti-inflammatory, anti-scarring, anti-antigenic, and anti-adhesive properties) relative to fresh (i.e., non-frozen) chorion or amnion-chorion.

In some embodiments, freezing the placenta results in the loss of metabolic activity in substantially all cells found in the chorion or amnion-chorion. In some embodiments, freezing the placenta results in the loss of metabolic activity in substantially all cells found in the chorion or amnion-chorion while maintaining or increasing the biological activity of the chorion or amnion-chorion chorion (e.g., its anti-inflammatory, anti-scarring, anti-antigenic, and anti-adhesive properties) relative to fresh (i.e., non-frozen) chorion or amnion-chorion.

In some embodiments, the placenta is dried. In some embodiments, drying the chorion or amnion-chorion kills substantially all cells found in the chorion or amnion-chorion. In some embodiments, drying the chorion or amnion-chorion results in the loss of metabolic activity in substantially all cells found in the chorion or amnion-chorion.

### Initial Processing of Placenta

All processing is done following Good Tissue Practices (GTP) to ensure that no contaminants are introduced into the tissue products.

The placenta is tested for HIV-1, HIV-2, HTLV-1, hepatitis B and C, West Nile virus, cytomegalovirus, human transmissible spongiform encephalopathy (e.g., Creutzfeldt-Jakob disease) and treponema pallidum using FDA licensed screening test. Any indication that the tissue is contaminated with HIV-1, HIV-2, HTLV-1, hepatitis B and C, West Nile virus, or cytomegalovirus results in the immediate quarantine and subsequent destruct of the tissue specimen.

Further, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and treponema pallidum results in the immediate quarantine and subsequent destruct of the tissue specimen.

In some embodiments, the placenta is frozen. In some embodiments, the placenta is fresh (i.e., not frozen). If the placenta is fresh (i.e., not frozen), it is processed as described below immediately.

### Further Processing

In some embodiments, the chorion or amnion-chorion is not separated from the placenta before further processing begins. In some embodiments, the chorion or amnion-chorion is separated from the placenta (generating isolated chorion or isolated amnion-chorion) before further processing begins,

In some embodiments, substantially all of the blood is removed from the placenta, isolated chorion, or isolated amnion-chorion. In some embodiments, some blood is removed from the placenta, chorion, or amnion-chorion. In some embodiments, the blood is not removed from the placenta, chorion, or amnion-chorion.

In some embodiments, the placenta, isolated chorion, or isolated amnion-chorion is washed with buffer with agitation to remove excess blood and tissue. In some embodiments, washing with agitation reduces the wash time.

In some embodiments, the placenta, isolated chorion, or isolated amnion-chorion is washed with an isotonic buffer or tissue culture media. In some embodiments, the placenta, isolated chorion, or isolated amnion-chorion is washed with saline. In some embodiments, the placenta, isolated chorion, or isolated amnion-chorion is washed with PBS. In some embodiments, the placenta, isolated chorion, or isolated amnion-chorion is washed with PBS1×. In some embodiments, the placenta, isolated chorion, or isolated amnion-chorion is washed with a TRIS-buffered saline. In some embodiments, the placenta, isolated chorion, or isolated amnion-chorion is washed with a HEPES-buffered saline. In some embodiments, the placenta, isolated chorion, or isolated amnion-chorion is washed with Ringer's solution. In some embodiments, the placenta, isolated chorion, or isolated amnion-chorion is washed with Hartmann's solution. In some embodiments, the placenta, isolated chorion, or isolated amnion-chorion is washed with EBSS. In some embodiments, the placenta, isolated chorion, or isolated amnion-chorion is washed with HBSS. In some embodiments, the placenta, isolated chorion, or isolated amnion-chorion is washed with Tyrode's Salt Solution. In some embodiments, the placenta, isolated chorion, or isolated amnion-chorion is washed with Gey's Balanced Salt Solution. In some embodiments, the placenta, isolated chorion, or isolated amnion-chorion is washed with DMEM. In some embodiments, the placenta, isolated chorion, or isolated amnion-chorion is washed with EMEM. In some embodiments, the placenta, isolated chorion, or isolated amnion-chorion is washed with GMEM. In some embodiments, the placenta, isolated chorion, or isolated amnion-chorion is washed with RPMI.

In some embodiments, the placenta, isolated chorion, or isolated amnion-chorion is washed with an isotonic buffer or tissue culture media, and any suitable antibiotic. In some embodiments, the antibiotic is ciprofloxacin, amphotericin B, penicillin, streptomycin, neomycin or a combination thereof. In some embodiments, the antibiotic is ciprofloxacin and amphotericin B. In some embodiments, the antibiotic is penicillin, streptomycin, neomycin, and amphotericin B.

### Processing to Generate Pulverized Tissue Product

Where the chorion or amnion-chorion is not separated from the placenta before the further processing steps, the chorion or amnion-chorion is isolated following the further processing steps, generating isolated chorion or isolated amnion-chorion.

In some embodiments the isolated chorion or isolated amnion-chorion is used to generate a pulverized tissue product. As used herein, “pulverized tissue product” means a tissue product comprising isolated chorion or isolated amnion-chorion that has been broken up. In some embodiments, the pulverized tissue product is a dry powder. In some embodiments, the pulverized tissue product is a solution, suspension or emulsion formed by mixing the pulverized tissue product with a carrier. In some embodiments, the pulverized tissue product is formulated into a cream, lotion, ointment, paste, gel, film, or paint. In some embodiments, the pulverized tissue product is contacted with a patch or wound dressing.

In some embodiments, the isolated chorion or isolated amnion-chorion is pulverized by any suitable method. In some embodiments, the isolated chorion or isolated amnion-chorion is pulverized by use of a pulverizer (e.g., a Bessman Tissue Pulverizer or a Covaris CryoPrep). In some embodiments, the isolated chorion or isolated amnion-chorion is pulverized by use of a tissue grinder (e.g., a Potter-Elvehjem grinder or a Wheaton Overhead Stirrer). In some embodiments, the isolated chorion or isolated amnion-chorion is pulverized by use of a sonicator. In some embodiments, the isolated chorion or isolated amnion-chorion is pulverized by use of a bead beater. In some embodiments, the isolated chorion or isolated amnion-chorion is pulverized by use of a freezer/mill (e.g., a SPEX SamplePrep Freezer/Mill). In some embodiments, the isolated chorion or isolated amnion-chorion is pulverized by use of a pestle and mortar. In some embodiments, the isolated chorion or isolated amnion-chorion is pulverized by manual use of a pestle and mortar.

In some embodiments, the isolated chorion or isolated amnion-chorion is optionally lyophilized before being pulverized. In some embodiments, the isolated chorion or isolated amnion-chorion is lyophilized by any suitable method (e.g., exposure to a liquid gas, placement in a freezer). In some embodiments, the isolated chorion or isolated amnion-chorion placed in the vacuum chamber of a lyophilization device until all or substantially all fluid (e.g., water) has been removed. In some embodiments, the isolated chorion or isolated amnion-chorion is lyophilized following freezing (e.g., exposure to a temperature below 0° C., −20° C., −40° C., −50° C., −60° C., −70° C., −75° C., −80° C., −90° C., −100° C.).

### Generation of Tissue Extracts

In some embodiments, an extract is made from the pulverized tissue product. In some embodiments, the pulverized tissue product is centrifuged to generate an extract (i.e., a chorion extract or an amnion-chorion extract). Any suitable method of centrifugation may be used. In some embodiments, the extract comprises the supernatant. In some embodiments, the extract comprises the precipitant. In some embodiments, the extract is subject to additional extraction methods (e.g., HABP affinity chromotography, or immunoaffinity chromatography).

In some embodiments, the method of making the extract comprises: (a) mixing the pulverized tissue product with cold PBS buffer without protease inhibitors, to generate a tissue product/PBS mixture, (b) centrifuging the tissue product/PBS mixture, and (c) isolating the extract, to generate an isolated extract. In some embodiments, the cold PBS buffer and tissue product are combined in a 1:1 ratio. In some embodiments, the tissue product/PBS mixture is centrifuged at 48,000×g 4° C. for 30 min.

In some embodiments, the method of making the extract further comprises purifying the extract. The number of purification steps depends on the desired purity. In some embodiments, the method of purifying the isolated extract comprises: (d) dissolving the isolated extract in CsCl/4M guanidine HCl at the initial density of 1.35 g/ml, to generate a CsCl mixture, (e) centrifuging the CsCl mixture at 125,000×g for 48 h at 15° C., to generate a first purified extract, (f) extracting the first purified extract and dialyzing it against distilled water to remove CsCl and guanidine HCl, to generate a dialysate. In some embodiments, the method of purifying the isolated extract further comprises (g) mixing the dialysate with 3 volumes of 95% (v/v) ethanol containing 1.3% (w/v) potassium acetate at 0° C. for 1 h, to generate a first dialysate/ethanol mixture, (h) centrifuging the first dialysate/ethanol mixture at 15,000×g, to generate a second purified extract, and (i) extracting the second purified extract. In some embodiments, the method of purifying the isolated extract further comprises: (j) washing the second purified extract with ethanol (e.g., 70% ethanol), to generate a second purified extract/ethanol mixture; (k) centrifuging the second purified extract/ethanol mixture, to generate a third purified extract; and (l) extracting the third purified extract.

### Generation of Homogenized Tissue Product

Disclosed herein, in some embodiments, is a homogenized tissue product, comprising: an isolated chorion or isolated amnion-chorion that is substantially free of cells with metabolic activity, wherein the natural biological activity of the isolated chorion or isolated amnion-chorion is substantially maintained. In some embodiments, the homogenized tissue product sheet is substantially free of HIV-1, HIV-2, HTLV-1, hepatitis B and C, West Nile virus, cytomegalovirus, human transmissible spongiform encephalopathy (e.g., Creutzfeldt-Jakob disease) and treponema pallidum. In some embodiments, the structural integrity of the isolated chorion or isolated amnion-chorion is substantially maintained.

Further disclosed herein, in certain embodiments, are methods of producing a homogenized tissue product, comprising: obtaining placenta, and separating the chorion or amnion-chorion from the rest of the placenta, wherein the natural biological activity of the chorion or amnion-chorion is substantially maintained. In some embodiments, the natural structural integrity of the chorion or amnion-chorion is substantially maintained.

Placenta is recovered from any suitable source (e.g., a hospital or tissue bank). Placenta can be obtained from any mammal, such as a human, non-human primate, cow or pig. The placenta may be frozen, previously frozen, or fresh (i.e., non-frozen).

Where the placenta is not processed into a pulverized tissue product immediately after it has been obtained, it is processed for storage (e.g., it is frozen or dried). In some embodiments, the placenta is frozen for storage. The placenta is frozen at any suitable temperature (e.g., −80° C.). In some embodiments, the placenta is frozen at −80° C. In some embodiments, the placenta is frozen until donor and specimen eligibility has been determined. In some embodiments, the placenta is placed in a cryo-preservative before being frozen.

In some embodiments, freezing the placenta kills substantially all cells found in the chorion or amnion-chorion. In some embodiments, freezing the placenta kills substantially all cells found in the chorion or amnion-chorion while maintaining or increasing the biological activity of the chorion or isolated chorion (e.g., its anti-inflammatory, anti-scarring, anti-antigenic, and anti-adhesive properties) relative to fresh (i.e., non-frozen) chorion or amnion-chorion.

In some embodiments, freezing the placenta results in the loss of metabolic activity in substantially all cells found in the chorion or amnion-chorion. In some embodiments, freezing the placenta results in the loss of metabolic activity in substantially all cells found in the chorion or amnion-chorion while maintaining or increasing the biological activity of the chorion or amnion-chorion chorion (e.g., its anti-inflammatory, anti-scarring, anti-antigenic, and anti-adhesive properties) relative to fresh (i.e., non-frozen) chorion or amnion-chorion.

In some embodiments, the placenta is dried. In some embodiments, drying the chorion or amnion-chorion kills substantially all cells found in the chorion or amnion-chorion. In some embodiments, drying the chorion or amnion-chorion results in the loss of metabolic activity in substantially all cells found in the chorion or amnion-chorion.

### Initial Processing of Placenta

All processing is done following Good Tissue Practices (GTP) to ensure that no contaminants are introduced into the tissue products.

The placenta is tested for HIV-1, HIV-2, HTLV-1, hepatitis B and C, West Nile virus, cytomegalovirus, human transmissible spongiform encephalopathy (e.g., Creutzfeldt-Jakob disease) and treponema pallidum using FDA licensed screening test. Any indication that the tissue is contaminated with HIV-1, HIV-2, HTLV-1, hepatitis B and C, West Nile virus, or cytomegalovirus results in the immediate quarantine and subsequent destruct of the tissue specimen.

Further, the donor's medical records are examined for risk factors for and clinical evidence of hepatitis B, hepatitis C, or HIV infection. Any indication that the donor has risk factors for, and/or clinical evidence of, infection with HIV-1, HIV-2, HTLV-1, hepatitis B and C, West Nile virus, cytomegalovirus, human transmissible spongiform encephalopathy (e.g., Creutzfeldt-Jakob disease) and treponema pallidum results in the immediate quarantine and subsequent destruct of the tissue specimen.

In some embodiments, the placenta is frozen. In some embodiments, the placenta is fresh (i.e., not frozen). If the placenta is fresh (i.e., not frozen), it is processed as described below immediately.

### Further Processing

In some embodiments, the chorion or amnion-chorion is not separated from the placenta before further processing begins. In some embodiments, the chorion or amnion-chorion is separated from the placenta (generating isolated chorion or isolated amnion-chorion) before further processing begins,

In some embodiments, substantially all of the blood is removed from the placenta, isolated chorion, or isolated amnion-chorion. In some embodiments, some blood is removed from the placenta, chorion, or amnion-chorion. In some embodiments, the blood is not removed from the placenta, chorion, or amnion-chorion.

In some embodiments, the placenta, isolated chorion, or isolated amnion-chorion is washed with buffer with agitation to remove excess blood and tissue. In some embodiments, washing with agitation reduces the wash time.

In some embodiments, the placenta, isolated chorion, or isolated amnion-chorion is washed with an isotonic buffer or tissue culture media. In some embodiments, the placenta, isolated chorion, or isolated amnion-chorion is washed with saline. In some embodiments, the placenta, isolated chorion, or isolated amnion-chorion is washed with PBS. In some embodiments, the placenta, isolated chorion, or isolated amnion-chorion is washed with PBS1×. In some embodiments, the placenta, isolated chorion, or isolated amnion-chorion is washed with a TRIS-buffered saline. In some embodiments, the placenta, isolated chorion, or isolated amnion-chorion is washed with a HEPES-buffered saline. In some embodiments, the placenta, isolated chorion, or isolated amnion-chorion is washed with Ringer's solution. In some embodiments, the placenta, isolated chorion, or isolated amnion-chorion is washed with Hartmann's solution. In some embodiments, the placenta, isolated chorion, or isolated amnion-chorion is washed with EBSS. In some embodiments, the placenta, isolated chorion, or isolated amnion-chorion is washed with HBSS. In some embodiments, the placenta, isolated chorion, or isolated amnion-chorion is washed with Tyrode's Salt Solution. In some embodiments, the placenta, isolated chorion, or isolated amnion-chorion is washed with Gey's Balanced Salt Solution. In some embodiments, the placenta, isolated chorion, or isolated amnion-chorion is washed with DMEM. In some embodiments, the placenta, isolated chorion, or isolated amnion-chorion is washed with EMEM. In some embodiments, the placenta, isolated chorion, or isolated amnion-chorion is washed with GMEM. In some embodiments, the placenta, isolated chorion, or isolated amnion-chorion is washed with RPMI.

In some embodiments, the placenta, isolated chorion, or isolated amnion-chorion is washed with an isotonic buffer or tissue culture media, and any suitable antibiotic. In some embodiments, the antibiotic is ciprofloxacin, amphotericin B, penicillin, streptomycin, neomycin or a combination thereof. In some embodiments, the antibiotic is ciprofloxacin and amphotericin B. In some embodiments, the antibiotic is penicillin, streptomycin, neomycin, and amphotericin B.

### Processing to Generate Homogenized Tissue Product

Where the chorion or amnion-chorion is not separated from the placenta before the further processing steps, the chorion or amnion-chorion is isolated following the further processing steps, generating isolated chorion or isolated amnion-chorion.

In some embodiments the isolated chorion or isolated amnion-chorion is used to generate a homogenized tissue product. As used herein, “homogenized tissue product” means a tissue product comprising isolated chorion or isolated amnion-chorion that has been broken up into particles that are of substantially uniform size. In some embodiments, the homogenized tissue product is a dry powder. In some embodiments, the homogenized tissue product is a solution, suspension or emulsion formed by mixing the homogenized tissue product with a carrier. In some embodiments, the homogenized tissue product is formulated into a cream, lotion, ointment, paste, gel, film or paint. In some embodiments, the homogenized tissue product is contacted with a patch or wound dressing.

In some embodiments, the isolated chorion or isolated amnion-chorion is homogenized by any suitable method. In some embodiments, the isolated chorion or isolated amnion-chorion is homogenized by use of a homogenizer (e.g., an ultrasonic homogenizer). In some embodiments, the isolated chorion or isolated amnion-chorion is homogenized by use of a sonicator. In some embodiments, the isolated chorion or isolated amnion-chorion is homogenized by use of a pulverizer, Warring blender, grinding mill, bead beater, or any combination thereof.

In some embodiments, the isolated chorion or isolated amnion-chorion is optionally lyophilized before being homogenized. In some embodiments, the isolated chorion or isolated amnion-chorion is lyophilized by any suitable method (e.g., exposure to a liquid gas, placement in a freezer). In some embodiments, the isolated chorion or isolated amnion-chorion placed in the vacuum chamber of a lyophilization device until all or substantially all fluid (e.g., water) has been removed. In some embodiments, the isolated chorion or isolated amnion-chorion is lyophilized following freezing (e.g., exposure to a temperature below 0° C., −20° C., −40° C., −50° C., −60° C., −70° C., −75° C., −80° C., −90° C., −100° C.).

### Generation of Tissue Extracts

In some embodiments, an extract is made from the homogenized tissue product. In some embodiments, the homogenized tissue product is centrifuged to generate an extract (i.e., a chorion extract or an amnion-chorion extract). Any suitable method of centrifugation may be used. In some embodiments, the extract comprises the supernatant. In some embodiments, the extract comprises the precipitant. In some embodiments, the extract is subject to additional extraction methods (e.g., HABP affinity chromotography, or immunoaffinity chromatography).

In some embodiments, the method of making the extract comprises: (a) mixing the homogenized tissue product with cold PBS buffer without protease inhibitors, to generate a tissue product/PBS mixture, (b) centrifuging the tissue product/PBS mixture, and (c) isolating the extract, to generate an isolated extract. In some embodiments, the cold PBS buffer and tissue product are combined in a 1:1 ratio. In some embodiments, the tissue product/PBS mixture is centrifuged at 48,000×g 4° C. for 30 min.

In some embodiments, the method of making the extract further comprises purifying the extract. The number of purification steps depends on the desired purity. In some embodiments, the method of purifying the isolated extract comprises: (d) dissolving the isolated extract in CsCl/4M guanidine HCl at the initial density of 1.35 g/ml, to generate a CsCl mixture, (e) centrifuging the CsCl mixture at 125,000×g for 48 h at 15° C., to generate a first purified extract, (f) extracting the first purified extract and dialyzing it against distilled water to remove CsCl and guanidine HCl, to generate a dialysate. In some embodiments, the method of purifying the isolated extract further comprises (g) mixing the dialysate with 3 volumes of 95% (v/v) ethanol containing 1.3% (w/v) potassium acetate at 0° C. for 1 h, to generate a first dialysate/ethanol mixture, (h) centrifuging the first dialysate/ethanol mixture at 15,000×g, to generate a second purified extract, and (i) extracting the second purified extract. In some embodiments, the method of purifying the isolated extract further comprises: (j) washing the second purified extract with ethanol (e.g., 70% ethanol), to generate a second purified extract/ethanol mixture; (k) centrifuging the second purified extract/ethanol mixture, to generate a third purified extract; and (l) extracting the third purified extract.

### Storage of Tissue Products

In some embodiments, a tissue product described herein (e.g., a flat tissue product sheet, a pulverized tissue product, or a homogenized tissue product is stored for later use. In some embodiments, storing a tissue product described herein (e.g., a flat tissue product sheet, a pulverized tissue product, or a homogenized tissue product) does not destroy the natural biological activity of the tissue product. In some embodiments, storing a tissue product described herein (e.g., a flat tissue product sheet, a pulverized tissue product, or a homogenized tissue product) does not destroy the natural structural integrity of the tissue product.

In some embodiments, the tissue product is stored in any suitable storage medium. In some embodiments, the storage medium is DMEM, Liebowitz's medium, MEM, NCTC, or any combination thereof.

In some embodiments, the storage medium comprises a high oncotic or hyperosmotic agent (also referred to as a “plasma expander”). In some embodiments, the hyperosmotic agent is propylene glycol, glycerol; sugars, such as glucose, sucrose, maltose, dextrose, and the like; dimethyl sulfoxide (DMSO); dimethylamine (DMA); polyvinylpyrrolidone (PVP); sorbitol, glutathione (GSH); ascorbic acid; rosmarinic acid (RO); riboflavin; dextran; albumin; trehalose; mannitol; or any combination thereof. The hyperosmotic agent maintains an optimal hydration state of the tissue product. Preferably, the hydration of the tissue product is maintained between about 60% and 90% hydration for the intended purpose. In some embodiments, the hyperosmotic agent makes up about 10% to 90% of the storage medium, preferably 10% to about 50%, and more preferably about 30% to about 50%. In some embodiments, the tissue product is stored in 10%, 20%, 30%, 40%, 50%, 60%, 70%, 80%, 90% or 100% glycerol. In some embodiments, the tissue product is stored in 10%, 20%, 30%, 40%, 50%, 60%, 70%, 80%, 90% or 100% propylene glycol.

In some embodiments, the tissue product is stored in 50% DMEM+50% Glycerol.

### Cryopreservation

In some embodiments, a tissue product described herein (e.g., a flat tissue product sheet, a pulverized tissue product, or a homogenized tissue product) is frozen for cryopreservation by any suitable method (e.g., exposure to a liquid gas, placement in a freezer). In some embodiments, cryopreserving a tissue product described herein (e.g., a flat tissue product sheet, a pulverized tissue product, or a homogenized tissue product) does not destroy the natural biological activity of the tissue product. In some embodiments, cryopreserving a tissue product described herein (e.g., a flat tissue product sheet, a pulverized tissue product, or a homogenized tissue product) does not destroy the natural structural integrity of the tissue product.

In some embodiments, a tissue product described herein (e.g., a flat tissue product sheet, a pulverized tissue product, or a homogenized tissue product) is exposed to a liquid gas (e.g., liquid nitrogen or liquid hydrogen). In some embodiments, tissue product disclosed herein is exposed to liquid nitrogen. In some embodiments, tissue product disclosed herein does not contact the liquid gas. In some embodiments, tissue product disclosed herein is placed in a container and the container is contacted with liquid gas. In some embodiments, tissue product disclosed herein is exposed to the liquid gas until the tissue product or chorion is frozen.

In some embodiments, tissue product disclosed herein is frozen by exposure to a temperature below about 0° C. In some embodiments, a tissue product described herein (e.g., a flat tissue product sheet, a pulverized tissue product, or a homogenized tissue product) is frozen by exposure to a temperature below about −20° C. In some embodiments, a tissue product described herein (e.g., a flat tissue product sheet, a pulverized tissue product, or a homogenized tissue product) is frozen by exposure to a temperature below about −40° C. In some embodiments, a tissue product described herein (e.g., a flat tissue product sheet, a pulverized tissue product, or a homogenized tissue product) is frozen by exposure to a temperature below about −50° C. In some embodiments, a tissue product described herein (e.g., a flat tissue product sheet, a pulverized tissue product, or a homogenized tissue product) is frozen by exposure to a temperature below about −60° C. In some embodiments, a tissue product described herein (e.g., a flat tissue product sheet, a pulverized tissue product, or a homogenized tissue product) is frozen by exposure to a temperature below about −70° C. In some embodiments, a tissue product described herein (e.g., a flat tissue product sheet, a pulverized tissue product, or a homogenized tissue product) is frozen by exposure to a temperature below about −75° C. In some embodiments, a tissue product described herein (e.g., a flat tissue product sheet, a pulverized tissue product, or a homogenized tissue product) is frozen by exposure to a temperature below about −80° C. In some embodiments, a tissue product described herein (e.g., a flat tissue product sheet, a pulverized tissue product, or a homogenized tissue product) is frozen by exposure to a temperature below about −90° C. In some embodiments, a tissue product described herein (e.g., a flat tissue product sheet, a pulverized tissue product, or a homogenized tissue product) is frozen by exposure to a temperature below about −100° C. In some embodiments, a tissue product described herein (e.g., a flat tissue product sheet, a pulverized tissue product, or a homogenized tissue product) is frozen by exposure to a liquid gas.

In some embodiments, a tissue product described herein (e.g., a flat tissue product sheet, a pulverized tissue product, or a homogenized tissue product) is placed in a suitable cryo-preservative. In some embodiments, the cryo-preservative comprises Glycerol, Propylene Glycol, DMSO, Trehalose, Mannitol, or a combination thereof. In some embodiments, the cryo-preservative comprises Glycerol, Propylene Glycol, or a combination.

### Lyophilization

In some embodiments, tissue product disclosed herein is lyophilized. In some embodiments, lyophilizing a tissue product described herein (e.g., a flat tissue product sheet, a pulverized tissue product, or a homogenized tissue product) does not destroy the natural biological activity of the tissue product. In some embodiments, lyophilizing a tissue product described herein (e.g., a flat tissue product sheet, a pulverized tissue product, or a homogenized tissue product) does not destroy the natural structural integrity of the tissue product.

In some embodiments, tissue product disclosed herein is lyophilized following freezing. In some embodiments, tissue product disclosed herein is lyophilized following freezing by any suitable method (e.g., exposure to a liquid gas, placement in a freezer).

In some embodiments, a frozen tissue product disclosed herein is placed in the vacuum chamber of a lyophilization device until all or substantially all fluid (e.g., water) has been removed.

### Sterilization

In some embodiments, a tissue product described herein (e.g., a flat tissue product sheet, a pulverized tissue product, or a homogenized tissue product) is subject to terminal sterilization by any suitable (e.g., medically acceptable) method. In some embodiments, a tissue product described herein (e.g., a flat tissue product sheet, a pulverized tissue product, or a homogenized tissue product) is exposed to gamma radiation for a period of time sufficient to sterilize the tissue product. In some embodiments, a tissue product described herein (e.g., a flat tissue product sheet, a pulverized tissue product, or a homogenized tissue product) is exposed to gamma radiation at 25 kGy for a period of time sufficient to sterilize the tissue product. In some embodiments, a tissue product described herein (e.g., a flat tissue product sheet, a pulverized tissue product, or a homogenized tissue product) is exposed to gamma radiation at 17-30 kGy for a period of time sufficient to sterilize the tissue product. In some embodiments, a tissue product described herein (e.g., a flat tissue product sheet, a pulverized tissue product, or a homogenized tissue product) is exposed to an electron beam for a period of time sufficient to sterilize the tissue product. In some embodiments, a tissue product described herein (e.g., a flat tissue product sheet, a pulverized tissue product, or a homogenized tissue product) is exposed to X-ray radiation for a period of time sufficient to sterilize the tissue product. In some embodiments, a tissue product described herein (e.g., a flat tissue product sheet, a pulverized tissue product, or a homogenized tissue product) is exposed to UV radiation for a period of time sufficient to sterilize the tissue product.

In some embodiments, a tissue product described herein (e.g., a flat tissue product sheet, a pulverized tissue product, or a homogenized tissue product) is placed in a suitable radio-protectant. In some embodiments, the radio-protectant comprises Glycerol, Propylene Glycol, DMSO, Trehalose, Mannitol, or a combination thereof. In some embodiments, the radio-protectant comprises Glycerol, Propylene Glycol, or a combination thereof.

### Rehydration

In some embodiments, dehydrated or lyophilized product tissue product disclosed herein is partially or fully rehydrated. In some embodiments, dehydrated or lyophilized product tissue product disclosed herein rehydrated by contacting the tissue product with a buffer or with water. In some embodiments, the tissue product is contacted with an isotonic buffer. In some embodiments, the tissue product is contacted with saline. In some embodiments, the tissue product is contacted with PBS. In some embodiments, the tissue product is contacted with Ringer's solution. In some embodiments, the tissue product is contacted with Hartmann's solution. In some embodiments, the tissue product is contacted with a TRIS-buffered saline. In some embodiments, the tissue product is contacted with a HEPES-buffered saline; 50% DMEM+50% Glycerol; 10%, 20%, 30%, 40%, 50%, 60%, 70%, 80%, 90% or 100% glycerol; and/or 10%, 20%, 30%, 40%, 50%, 60%, 70%, 80%, 90% or 100% propylene glycol.

### Tissue Product Formulations

In some embodiments, a tissue product disclosed herein is a solution, suspension or emulsion formed by mixing the tissue product with a carrier. In some embodiments, the tissue product is formulated for topical administration. In some embodiments, the tissue product is formulated for injection.

Tissue product formulations disclosed herein are formulated in any suitable manner. Any suitable technique, carrier, and/or excipient is contemplated for use with the LPA receptor antagonists disclosed herein.

### Creams and Lotions

Disclosed herein, in certain embodiments, is a tissue product formulation, wherein a tissue product described herein (e.g., a flat tissue product sheet, a pulverized tissue product, or a homogenized tissue product) is formulated as a cream. In certain instances, creams are semisolid (e.g., soft solid or thick liquid) formulations that include a tissue product described herein (e.g., a flat tissue product sheet, a pulverized tissue product, or a homogenized tissue product) dispersed in an oil-in-water emulsion or a water-in-oil emulsion.

Disclosed herein, in certain embodiments, is a tissue product formulation wherein a tissue product described herein (e.g., a flat tissue product sheet, a pulverized tissue product, or a homogenized tissue product) is formulated as a lotion. In certain instances, lotions are fluid emulsions (e.g., oil-in-water emulsions or a water-in-oil emulsion). In some embodiments, the hydrophobic component of a lotion and/or cream is derived from an animal (e.g., lanolin, cod liver oil, and ambergris), plant (e.g., safflower oil, castor oil, coconut oil, cottonseed oil, menhaden oil, palm kernel oil, palm oil, peanut oil, soybean oil, rapeseed oil, linseed oil, rice bran oil, pine oil, sesame oil, or sunflower seed oil), or petroleum (e.g., mineral oil, or petroleum jelly).

### Ointments

Disclosed herein, in certain embodiments, is a tissue product formulation, wherein a tissue product described herein (e.g., a flat tissue product sheet, a pulverized tissue product, or a homogenized tissue product) is formulated as an ointment. In certain instances, ointments are semisolid preparations that soften or melt at body temperature.

### Pastes

Disclosed herein, in certain embodiments, is a tissue product formulation, wherein a tissue product described herein (e.g., a flat tissue product sheet, a pulverized tissue product, or a homogenized tissue product) is formulated as a paste. In certain instances, pastes contain at least 20% solids. In certain instances, pastes are ointments that do not flow at body temperature.

### Gels and Films

Disclosed herein, in certain embodiments, is a tissue product formulation, wherein a tissue product described herein (e.g., a flat tissue product sheet, a pulverized tissue product, or a homogenized tissue product) is formulated as a gel. In certain instances, gels are semisolid (or semi-rigid) systems consisting of dispersions of large organic molecules dispersed in a liquid. In certain instances, gels are water-soluble and are removed using warm water or saline.

In certain instances, in the treatment of dermal lesions, contacting lesions with a dressing can often disturb injured tissues. The removal of many dressings for wounds such as burns surface lesions that involve a significant area of the skin can cause significant pain and often can re-open at least portions of partially healed wounds. In some instances, the formulations described herein are applied as a liquid to the affected area and the liquid gels as a film on the affected area. In some instances the film is a water soluble film and can be removed with water or a mild aqueous detergent, avoiding pain and discomfort associated with the removal of wound dressings. In certain instances, the formulation described herein is a dermal film comprising a flexible film made of a polyalkyloxazoline. In some instances, the film has a structural layer made of a polyalkyloxazoline and a pressure sensitive adhesive layer that keeps the film in place.

### Sticks

Disclosed herein, in certain embodiments, is a tissue product formulation, wherein a tissue product described herein (e.g., a flat tissue product sheet, a pulverized tissue product, or a homogenized tissue product) is formulated as a stick. In certain instances, sticks are solid dosage forms that melt at body temperature. In some embodiments, a stick comprises a wax, a polymer, a resin, dry solids fused into a firm mass, and/or fused crystals. In some embodiments, a tissue product described herein (e.g., a flat tissue product sheet, a pulverized tissue product, or a homogenized tissue product) is formulated as a styptic pencil (i.e., a stick prepared by (1) heating crystals until they lose their water of crystallization and become molten, and (2) pouring the molten crystals into molds and allowing them to harden). In some embodiments, a a tissue product described herein (e.g., a flat tissue product sheet, a pulverized tissue product, or a homogenized tissue product) is formulated as a stick wherein the stick comprises a wax (e.g., the wax is melted and poured into appropriate molds in which they solidify in stick form).

In some embodiments, a tissue product described herein (e.g., a flat tissue product sheet, a pulverized tissue product, or a homogenized tissue product) is formulated as a stick wherein the stick comprises a melting base (i.e., a base that softens at body temperature). Examples of melting bases include, but are not limited to, waxes, oils, polymers and gels. In some embodiments, a a tissue product described herein (e.g., a flat tissue product sheet, a pulverized tissue product, or a homogenized tissue product) is formulated as a stick wherein the stick comprises a moisten base (i.e., a base that is activated by the addition of moisture).

### Patches

In some embodiments, a tissue product formulation disclosed is administered via a patch. In some embodiments, a tissue product described herein is dissolved and/or dispersed in a polymer or an adhesive. In some embodiments, a film, a patch disclosed herein is constructed for continuous, pulsatile, or on demand delivery of a tissue product described herein (e.g., a flat tissue product sheet, a pulverized tissue product, or a homogenized tissue product).

### Wound Dressings

In some embodiments, a tissue product formulation disclosed herein is administered with (or via) a wound dressing. Wound dressings include, but are not limited to gauzes, transparent film dressings, hydrogels, polyurethane foam dressings, hydrocolloids and alginates. In certain instances, wound dressings promote wound healing. In some instances wound dressings reduce or inhibit aberrant wound healing.

### Miscellaneous Formulations

In some embodiments, the formulations and compositions disclosed herein are administered as a dermal paint. As used herein, paints (also known as film formers) are solutions comprised of a solvent, a monomer or polymer, an active agent, and optionally one or more pharmaceutically-acceptable excipients. After application to a tissue, the solvent evaporates leaving behind a thin coating comprised of the monomers or polymers, and the active agent. The coating protects active agents and maintains them in an immobilized state at the site of application. This decreases the amount of active agent which may be lost and correspondingly increases the amount delivered to the affected area of the skin of an individual. By way of non-limiting example, paints include collodions (e.g. Flexible Collodion, USP), and solutions comprising saccharide siloxane copolymers and a cross-linking agent. Collodions are ethyl ether/ethanol solutions containing pyroxylin (a nitrocellulose). After application, the ethyl ether/ethanol solution evaporates leaving behind a thin film of pyroxylin. In solutions comprising saccharide siloxane copolymers, the saccharide siloxane copolymers form the coating after evaporation of the solvent initiates the cross-linking of the saccharide siloxane copolymers.

In certain embodiments, the formulations described herein comprise tissue products that are optionally incorporated within controlled release particles, lipid complexes, liposomes, nanoparticles, microspheres, microparticles, nanocapsules or other agents which enhance or facilitate localized delivery to the skin. An example of a conventional microencapsulation process for pharmaceutical preparations is shown in U.S. Pat. No. 3,737,337 incorporated herein by reference for such disclosure.

In some instances, a formulation described herein is a liposomal formulation. Liposomes are prepared by introducing an aqueous buffer into a mixture of phospholipid and organic solvent and the organic solvent is subsequently removed by evaporation under reduced pressure. An example of a liposomal preparation is described in Proc. Natl. Acad. Sci. 1978, 75, 4194-98, incorporated herein by reference for such disclosure. Liposomes are fractionated according to their particle sizes by size exclusion chromatography (SEC). The subfractions of liposomes are further sized by photon correlation spectroscopy (PCS) for their particle sizes. Enzymatic assays (e.g., phosphatidylcholine (PC) assay) are used to analyze lipid contents of liposomes.

### Dermatological Excipients

Disclosed herein, in certain embodiments, is a tissue product formulation comprising isolated chorion or isolated amnion-chorion, wherein the formulation comprises a carrier. Suitable carriers include water, hyaluronan, collagen, ethanol, polyols (propyleneglycol, polyethylene-glycol, glycerol, cremophor and the like), vegetable oils (such as olive oil), injectable organic esters (e.g., ethyl oleate), fatty oils (e.g., sesame oil), and synthetic fatty acid esters (e.g., ethyl oleate or triglycerides).

### Penetration Enhancers

Disclosed herein, in certain embodiments, is a tissue product formulation wherein the formulation comprises a penetration enhancer. Penetration enhancers include, but are not limited to, sodium lauryl sulfate, sodium laurate, polyoxyethylene-20-cetyl ether, laureth-9, sodium dodecylsulfate, dioctyl sodium sulfosuccinate, polyoxyethylene-9-lauryl ether (PLE), Tween 80, nonylphenoxypolyethylene (NP-POE), polysorbates, sodium glycocholate, sodium deoxycholate, sodium taurocholate, sodium taurodihydrofusidate, sodium glycodihydrofusidate, oleic acid, caprylic acid, mono- and di-glycerides, lauric acids, acylcholines, caprylic acids, acylcarnitines, sodium caprates, EDTA, citric acid, salicylates, DMSO, decylmethyl sulfoxide, ethanol, isopropanol, propylene glycol, polyethylene glycol, glycerol, propanediol, and diethylene glycol monoethyl ether. In certain embodiments, the formulations described herein are designed for minimal systemic exposure and include, for example, low amounts of penetration enhancers.

### Gelling Agents

Disclosed herein, in certain embodiments, is a tissue product formulation wherein the formulation comprises a gelling (or thickening) agent. In some embodiments, a formulation disclosed herein further comprises from about 0.1% to about 5%, from about 0.1% to about 3%, or from about 0.25% to about 2%, of a gelling agent. In certain embodiments, the viscosity of a formulation disclosed herein is in the range from about 100 to about 500,000 cP, about 100 cP to about 1,000 cP, about 500 cP to about 1500 cP, about 1000 cP to about 3000 cP, about 2000 cP to about 8,000 cP, about 4,000 cP to about 10,000 cP, about 10,000 cP to about 50,000 cP. Suitable gelling agents for use in preparation of the gel formulation include, but are not limited to, celluloses, cellulose derivatives, cellulose ethers (e.g., carboxymethylcellulose, ethylcellulose, hydroxyethylcellulose, hydroxymethylcellulose, hydroxypropylmethylcellulose, hydroxypropylcellulose, methylcellulose), guar gum, xanthan gum, locust bean gum, alginates (e.g., alginic acid), silicates, starch, tragacanth, carboxyvinyl polymers, carrageenan, paraffin, petrolatum, acacia (gum arabic), agar, aluminum magnesium silicate, sodium alginate, sodium stearate, bladderwrack, bentonite, carbomer, carrageenan, carbopol, xanthan, cellulose, microcrystalline cellulose (MCC), ceratonia, chondrus, dextrose, furcellaran, gelatin, ghatti gum, guar gum, hectorite, lactose, sucrose, maltodextrin, mannitol, sorbitol, honey, maize starch, wheat starch, rice starch, potato starch, gelatin, sterculia gum, polyethylene glycol (e.g. PEG 200-4500), gum tragacanth, ethyl cellulose, ethylhydroxyethyl cellulose, ethylmethyl cellulose, methyl cellulose, hydroxyethyl cellulose, hydroxyethylmethyl cellulose, hydroxypropyl cellulose, poly(hydroxyethyl methacrylate), oxypolygelatin, pectin, polygeline, povidone, propylene carbonate, methyl vinyl ether/maleic anhydride copolymer (PVM/MA), poly(methoxyethyl methacrylate), poly(methoxyethoxyethyl methacrylate), hydroxypropyl cellulose, hydroxypropylmethyl-cellulose (HPMC), sodium carboxymethyl-cellulose (CMC), silicon dioxide, polyvinylpyrrolidone (PVP: povidone), or combinations thereof.

Gels include a single-phase or a two-phase system. A single-phase gel consists of organic macromolecules distributed uniformly throughout a liquid in such a manner that no apparent boundaries exist between the dispersed macromolecules and the liquid. Some single-phase gels are prepared from synthetic macromolecules (e.g., carbomer) or from natural gums, (e.g., tragacanth). In some embodiments, single-phase gels are generally aqueous, but will also be made using alcohols and oils. Two-phase gels consist of a network of small discrete particles.

Gels can also be classified as being hydrophobic or hydrophilic. In certain embodiments, the base of a hydrophobic gel consists of a liquid paraffin with polyethylene or fatty oils gelled with colloidal silica, or aluminum or zinc soaps. In contrast, the base of hydrophobic gels usually consists of water, glycerol, or propylene glycol gelled with a suitable gelling agent (e.g., tragacanth, starch, cellulose derivatives, carboxyvinylpolymers, and magnesium-aluminum silicates).

Suitable agents for use in formulations that are applied as liquids and gel upon application to the skin into a film include but are not limited to polymers composed of polyoxypropylene and polyoxyethylene that are known to form thermoreversible gels when incorporated into aqueous solutions. These polymers have the ability to change from the liquid state to the gel state at temperatures close to body temperature, therefore allowing useful formulations that are applied as gels and/or films to the affected area. Examples of polymers that gel at body temperature and are used in gels and/or films described herein include and are not limited to poloxamers (e.g., PLURONICS F68®, F88®, F108®, and F127®, which are block copolymers of ethylene oxide and propylene oxide). The liquid state-to-gel state phase transition is dependent on the polymer concentration and the ingredients in the solution.

### Adhesives

In some instances, the formulations described herein comprise pressure sensitive adhesives (e.g., polyalkyloxazoline polymers) and allow for application of an adhesive film to an affected area of skin.

### Emollients

Disclosed herein, in certain embodiments, is a tissue product formulation wherein the formulation comprises an emollient. Emollients include, but are not limited to, castor oil esters, cocoa butter esters, safflower oil esters, cottonseed oil esters, corn oil esters, olive oil esters, cod liver oil esters, almond oil esters, avocado oil esters, palm oil esters, sesame oil esters, squalene esters, kikui oil esters, soybean oil esters, acetylated monoglycerides, ethoxylated glyceryl monostearate, hexyl laurate, isohexyl laurate, isohexyl palmitate, isopropyl palmitate, methyl palmitate, decyloleate, isodecyl oleate, hexadecyl stearate decyl stearate, isopropyl isostearate, methyl isostearate, diisopropyl adipate, diisohexyl adipate, dihexyldecyl adipate, diisopropyl sebacate, lauryl lactate, myristyl lactate, and cetyl lactate, oleyl myristate, oleyl stearate, and oleyl oleate, pelargonic acid, lauric acid, myristic acid, palmitic acid, stearic acid, isostearic acid, hydroxystearic acid, oleic acid, linoleic acid, ricinoleic acid, arachidic acid, behenic acid, erucic acid, lauryl alcohol, myristyl alcohol, cetyl alcohol, hexadecyl alcohol, stearyl alcohol, isostearyl alcohol, hydroxystearyl alcohol, oleyl alcohol, ricinoleyl alcohol, behenyl alcohol, erucyl alcohol, 2-octyl dodecanyl alcohol, lanolin and lanolin derivatives, beeswax, spermaceti, myristyl myristate, stearyl stearate, carnauba wax, candelilla wax, lecithin, and cholesterol.

### Miscellaneous Excipients

In certain embodiments, a formulation comprising a tissue product comprises additional excipients such as, by way of example, abrasives, absorbents, anticaking agents, astringents, essential oils, fragrances, skin-conditioning agents, skin healing agents, skin protectants (e.g., sunscreens, or ultraviolet light absorbers or scattering agents), skin soothing agents, or combinations thereof.

### Methods of Use

Disclosed herein, in certain embodiments, are methods of using a tissue product described herein (e.g., a flat tissue product sheet, a pulverized tissue product, or a homogenized tissue product).

In some embodiments, a tissue product described herein (e.g., a flat tissue product sheet, a pulverized tissue product, or a homogenized tissue product) is used to inhibit at least one of the following: pain, scarring, inflammation, adhesive or angiogenesis. In some embodiments, a tissue product described herein (e.g., a flat tissue product sheet, a pulverized tissue product, or a homogenized tissue product) is used to promote wound healing. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) is used as a covering (e.g., a wound covering). In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) is used to promote wound repair. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) is used as a barrier to adhesive. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion. In certain instances, the chorion and/or amnion-chorion comprises proteins, glycans, protein-glycan complexes (e.g., a complex of hyaluronic acid and a heavy chain of IαI and PTX3) and enzymes such as TSG-6 that promote tissue repair.

In some embodiments, a tissue product described herein (e.g., a flat tissue product sheet, a pulverized tissue product, or a homogenized tissue product) comprises chorion or amnion-chorion as a scaffold, and a plurality of cells integrated into the scaffold. In some embodiments, the cells are embryonic stem cells, mesenchymal stem cells or adult lineage-committed stem cells or differentiated epidermal cells (e.g., to treat a burn or a surgical incision in the skin). In some embodiments, the cells are mesothelial cells (e.g., to treat to a wound (e.g., surgical incision) in an internal organ).

### Injured Tissue Repair and Supplementation

Disclosed herein, in certain embodiments, is the use of a tissue product described herein (e.g., a flat tissue product sheet, a pulverized tissue product, or a homogenized tissue product) for repairing, reconstructing, replacing, or supplementing a recipient's damaged, compromised, or missing recipient tissue. In some embodiments, a tissue product described herein (e.g., a flat tissue product sheet, a pulverized tissue product, or a homogenized tissue product) is used as a wound covering or is used to facilitate wound repair. In some embodiments, the use is a homologous use (e.g., a functional homologous use or a structural homologous use). In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, the recipient tissue was damaged, compromised, or lost due to an injury (e.g., a burn; a surgical incision; an area of necrosis resulting from an infection, trauma, or a toxin; a laceration). In some embodiments, the recipient tissue was damaged, compromised, or lost due to a burn. In some embodiments, the recipient tissue was damaged, compromised, or lost due to a wound (e.g., an incision, laceration, abrasion). In some embodiments, the recipient tissue was damaged, compromised, or lost due to necrosis. In some embodiments, the recipient tissue was damaged, compromised, or lost due to ulceration.

In some embodiments, a tissue product described herein (e.g., a flat tissue product sheet, a pulverized tissue product, or a homogenized tissue product) comprises chorion or amnion-chorion. In certain instances, the chorion and/or amnion-chorion comprises proteins, glycans, protein-glycan complexes (e.g., a complex of hyaluronic acid and a heavy chain of IαI and PTX3) and enzymes such as TSG-6 that promote tissue repair.

In some embodiments, a tissue product described herein (e.g., a flat tissue product sheet, a pulverized tissue product, or a homogenized tissue product) comprises chorion or amnion-chorion as a scaffold, and a plurality of cells integrated into the scaffold. In some embodiments, the cells are epidermal cells (e.g., to treat a burn or a surgical incision in the skin). In some embodiments, the cells are mesothelial cells (e.g., to treat to a wound (e.g., surgical incision) in an internal organ).

### Burns

In some embodiments, a tissue product described herein (e.g., a flat tissue product sheet, a pulverized tissue product, or a homogenized tissue product) is used as a protective graft over a burn. In some embodiments, a tissue product described herein (e.g., a flat tissue product sheet, a pulverized tissue product, or a homogenized tissue product) is used as a protective graft over a first degree burn. In some embodiments, a tissue product described herein (e.g., a flat tissue product sheet, a pulverized tissue product, or a homogenized tissue product) is used as a protective graft over a second degree burn. In some embodiments, a tissue product described herein (e.g., a flat tissue product sheet, a pulverized tissue product, or a homogenized tissue product) is used as a protective graft over a third degree burn. In some embodiments, a protective graft comprising a tissue product described herein (e.g., a flat tissue product sheet, a pulverized tissue product, or a homogenized tissue product) as described herein is placed on a burn.

In some embodiments, the protective graft comprises chorion or amnion-chorion. In some embodiments, the protective graft comprises chorion or amnion-chorion as a scaffold, and a plurality of epidermal cells integrated into the scaffold.

### Wounds

In some embodiments, a tissue product described herein (e.g., a flat tissue product sheet, a pulverized tissue product, or a homogenized tissue product) is used as a protective graft over a wound (e.g., an incision, laceration, abrasion, ulcer, puncture, penetration).

In some embodiments, a protective graft comprising a tissue product described herein (e.g., a flat tissue product sheet, a pulverized tissue product, or a homogenized tissue product) is placed on wound. In some embodiments, the protective graft comprises chorion or amnion-chorion. In some embodiments, the protective graft comprises chorion or amnion-chorion as a scaffold, and a plurality of epithelial cells (e.g., epidermal and/or mesothelial cells) integrated into the scaffold.

In some embodiments, a tissue product described herein (e.g., a flat tissue product sheet, a pulverized tissue product, or a homogenized tissue product) is used as a covering over an incision in an organ (e.g., the skin, brain, stomach, kidneys, liver, intestines, lungs, bladder, trachea, esophagus, vagina, ureter, and blood vessel walls). In some embodiments, a tissue product described herein (e.g., a flat tissue product sheet, a pulverized tissue product, or a homogenized tissue product) is placed on a surgical incision. In some embodiments, a tissue product described herein (e.g., a flat tissue product sheet, a pulverized tissue product, or a homogenized tissue product) is used to repair or supplement tissue following colon resection. In some embodiments, a tissue product described herein (e.g., a flat tissue product sheet, a pulverized tissue product, or a homogenized tissue product) is used to repair or supplement tissue following gastrectomy. In some embodiments, a tissue product described herein (e.g., a flat tissue product sheet, a pulverized tissue product, or a homogenized tissue product) is used to repair or supplement tissue following breast surgery (e.g., breast reduction surgery, breast augmentation surgery, and mastectomy). In some embodiments, tissue product disclosed herein comprises chorion or amnion-chorion as a scaffold, and a plurality of epithelial cells (e.g., epidermal and/or mesothelial cells) integrated into the scaffold.

In some embodiments, a tissue product described herein (e.g., a flat tissue product sheet, a pulverized tissue product, or a homogenized tissue product) is used as a covering over an incision in the skin (e.g., an incision to the epidermis, dermis, and/or hypodermis). In some embodiments, a tissue product described herein (e.g., a flat tissue product sheet, a pulverized tissue product, or a homogenized tissue product) is used to repair or supplement the skin following hemorrhoid surgery.

### Necrosis

In some embodiments, a tissue product described herein (e.g., a flat tissue product sheet, a pulverized tissue product, or a homogenized tissue product) is used as a protective graft over an area of necrotic tissue (e.g., from an infection). In some embodiments, a tissue product described herein (e.g., a flat tissue product sheet, a pulverized tissue product, or a homogenized tissue product) is used as a protective graft over an area of necrotic skin. In some embodiments, a protective graft comprising a tissue product described herein (e.g., a flat tissue product sheet, a pulverized tissue product, or a homogenized tissue product) is placed on an area of necrotic tissue. In some embodiments, the protective graft comprises chorion or amnion-chorion. In some embodiments, the protective graft comprises chorion or amnion-chorion as a scaffold, and a plurality of epidermal and/or mesothelial cells integrated into the scaffold.

### Ulcer

In some embodiments, a tissue product described herein (e.g., a flat tissue product sheet, a pulverized tissue product, or a homogenized tissue product) is used as a protective covering over an ulcer (e.g., a diabetic/neuropathic foot ulcer, decubitis ulcer, sickle cell ulcer or an arterial insufficiency ulcer). In some embodiments, the protective covering comprises chorion or amnion-chorion as a scaffold, and a plurality of epidermal and/or mesothelial cells integrated into the scaffold. In some embodiments, a protective covering is placed on an ulcer.

In some embodiments, the ulcer is a leg ulcer (e.g., a diabetic foot ulcer or an arterial insufficiency ulcer). In some embodiments, treating a foot ulcer comprises (a) preparing the wound (e.g., debriding the wound); and (b) placing a tissue product described herein (e.g., a flat tissue product sheet, a pulverized tissue product, or a homogenized tissue product) on the wound. In some embodiments, treating a foot ulcer comprises (a) preparing the wound (e.g., debriding the wound); (b) placing a tissue product described herein (e.g., a flat tissue product sheet, a pulverized tissue product, or a homogenized tissue product) on the wound; and (c) covering a tissue product described herein (e.g., a flat tissue product sheet, a pulverized tissue product, or a homogenized tissue product) with a protective barrier (e.g., a silvercell dressing, metipel, gauze, or a bandage).

In some embodiments, the ulcer is a venous stasis (VS) ulcer. In some embodiments, treating a VS ulcer comprises (a) preparing the wound (e.g., debriding the wound); and (b) placing a tissue product described herein (e.g., a flat tissue product sheet, a pulverized tissue product, or a homogenized tissue product) on the wound. In some embodiments, treating a VS ulcer comprises (a) preparing the wound (e.g., debriding the wound); (b) placing a tissue product described herein (e.g., a flat tissue product sheet, a pulverized tissue product, or a homogenized tissue product) on the wound; and (c) covering a tissue product described herein (e.g., a flat tissue product sheet, a pulverized tissue product, or a homogenized tissue product) with a protective barrier (e.g., a silvercell dressing, metipel, gauze, or a bandage). In some embodiments, treating a VS ulcer further comprises compression therapy (e.g. compression bandage or stocking).

In some embodiments, the ulcer is a corneal ulcer (i.e., ulcerative keratitis). In some embodiments, treating a corneal ulcer comprises (a) preparing the wound (e.g., debriding the wound); and (b) placing a tissue product described herein (e.g., a flat tissue product sheet, a pulverized tissue product, or a homogenized tissue product) on the wound. In some embodiments, treating a corneal ulcer comprises (a) preparing the wound (e.g., debriding the wound); (b) placing a tissue product described herein (e.g., a flat tissue product sheet, a pulverized tissue product, or a homogenized tissue product) on the wound; and (c) covering a tissue product described herein (e.g., a flat tissue product sheet, a pulverized tissue product, or a homogenized tissue product) with a protective barrier (e.g., a contact lens or a bandage).

### Soft Tissue Uses

Disclosed herein, in certain embodiments, is the use of a tissue product described herein (e.g., a flat tissue product sheet, a pulverized tissue product, or a homogenized tissue product) for repairing, reconstructing, replacing, or supplementing a recipient's damaged, compromised, or missing soft tissue (e.g., tendons). In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion. In certain instances, the tissue product comprises proteins, glycans, protein-glycan complexes (e.g., a complex of hyaluronic acid and a heavy chain of IαI and PTX3) and enzymes such as TSG-6 that promote tissue repair.

In some embodiments, a tissue product described herein (e.g., a flat tissue product sheet, a pulverized tissue product, or a homogenized tissue product) comprises chorion or amnion-chorion as a scaffold, and a plurality of cells integrated into the scaffold. In some embodiments, the cells are epidermal cells (e.g., to treat a burn or a surgical incision in the skin). In some embodiments, the cells are mesothelial cells (e.g., to treat to a wound (e.g., surgical incision) in an internal organ).

In some embodiments, a tissue product described herein (e.g., a flat tissue product sheet, a pulverized tissue product, or a homogenized tissue product) is used as a covering over an incision in soft tissue (e.g., eyelids form the tissue plane between different layers of soft tissue).

In some embodiments, a tissue product described herein (e.g., a flat tissue product sheet, a pulverized tissue product, or a homogenized tissue product) is used as structural (tectonic) support for soft tissue.

In some embodiments, a tissue product described herein (e.g., a flat tissue product sheet, a pulverized tissue product, or a homogenized tissue product) prevents adhesive in joint or tendon repairs.

In some embodiments, a tissue product described herein (e.g., a flat tissue product sheet, a pulverized tissue product, or a homogenized tissue product) is used in the repair a tendon or joint (such as rotator cuff repairs, hand tendon repairs). In some embodiments, a tissue product described herein (e.g., a flat tissue product sheet, a pulverized tissue product, or a homogenized tissue product) is used as a patch over a tendon (e.g., a tendon that has been torn or a tendon that has been sutured) or joint. In some embodiments, a tissue product described herein (e.g., a flat tissue product sheet, a pulverized tissue product, or a homogenized tissue product) is used to reconstruct a tendon. In some embodiments, a tissue product described herein (e.g., a flat tissue product sheet, a pulverized tissue product, or a homogenized tissue product) is used to augment a tendon or joint. In some embodiments, a tissue product described herein (e.g., a flat tissue product sheet, a pulverized tissue product, or a homogenized tissue product) is used to reinforce a tendon or joint. In some embodiments, a tissue product described herein (e.g., a flat tissue product sheet, a pulverized tissue product, or a homogenized tissue product) is used to prevent adhesive of a healing tendon to surrounding tissue, tendons or joints. In some embodiments, a tissue product described herein (e.g., a flat tissue product sheet, a pulverized tissue product, or a homogenized tissue product) is used to prevent the formation of scar tissue on a tendon.

In some embodiments, a tissue product described herein (e.g., a flat tissue product sheet, a pulverized tissue product, or a homogenized tissue product) is used to augment smaller tendons and ligaments of the foot and ankle, including the posterior tibial tendon, the personneal tendons, the flexor and extensor tendons, and the ligaments of the lateral ankle complex. In some embodiments, a tissue product described herein (e.g., a flat tissue product sheet, a pulverized tissue product, or a homogenized tissue product) is used to reinforce primary repair of the quadriceps and patellar tendons surrounding the knee. In some embodiments, a tissue product described herein (e.g., a flat tissue product sheet, a pulverized tissue product, or a homogenized tissue product) is used as a periosteal patch for bone graft in joint replacement. In some embodiments, a tissue product described herein (e.g., a flat tissue product sheet, a pulverized tissue product, or a homogenized tissue product) is used augment deficient hip and knee capsular tissue following total joint revision surgery.

In some embodiments, a tissue product described herein (e.g., a flat tissue product sheet, a pulverized tissue product, or a homogenized tissue product) is used in the repair of a torn rotator cuff. In some embodiments, a tissue product described herein (e.g., a flat tissue product sheet, a pulverized tissue product, or a homogenized tissue product) is used as a patch over a rotator cuff muscle or tendon (e.g., the supraspinatus tendon). In some embodiments, a tissue product described herein (e.g., a flat tissue product sheet, a pulverized tissue product, or a homogenized tissue product) is used to reconstruct a rotator cuff muscle or tendon (e.g., the supraspinatus tendon). In some embodiments, a tissue product described herein (e.g., a flat tissue product sheet, a pulverized tissue product, or a homogenized tissue product) is used to augment a rotator cuff muscle or tendon (e.g., the supraspinatus tendon). In some embodiments, a tissue product described herein (e.g., a flat tissue product sheet, a pulverized tissue product, or a homogenized tissue product) is used to reinforce a rotator cuff muscle or tendon (e.g., the supraspinatus tendon). In some embodiments, a tissue product described herein (e.g., a flat tissue product sheet, a pulverized tissue product, or a homogenized tissue product) is used to prevent adhesive of soft tissue to a rotator cuff muscle or tendon (e.g., the supraspinatus tendon).

In some embodiments, a tissue product described herein (e.g., a flat tissue product sheet, a pulverized tissue product, or a homogenized tissue product) is used in the repair gingiva. In some embodiments, a tissue product described herein (e.g., a flat tissue product sheet, a pulverized tissue product, or a homogenized tissue product) is used in the repair gingival recession. In some embodiments, a tissue product described herein (e.g., a flat tissue product sheet, a pulverized tissue product, or a homogenized tissue product) is used as a patch over gingiva. In some embodiments, a tissue product described herein (e.g., a flat tissue product sheet, a pulverized tissue product, or a homogenized tissue product) is used as a patch over an exposed tooth root surface. In some embodiments, a tissue product described herein (e.g., a flat tissue product sheet, a pulverized tissue product, or a homogenized tissue product) is used to reconstruct gingiva. In some embodiments, a tissue product described herein (e.g., a flat tissue product sheet, a pulverized tissue product, or a homogenized tissue product) is used to augment gingiva. In some embodiments, a tissue product described herein (e.g., a flat tissue product sheet, a pulverized tissue product, or a homogenized tissue product) is used to reinforce gingiva. In some embodiments, a tissue product described herein (e.g., a flat tissue product sheet, a pulverized tissue product, or a homogenized tissue product) is used to prevent adhesive of soft tissue to gingiva.

In some embodiments, a tissue product described herein (e.g., a flat tissue product sheet, a pulverized tissue product, or a homogenized tissue product) is used as a protective graft over an incision or tear in the fascia. In some embodiments, a tissue product described herein (e.g., a flat tissue product sheet, a pulverized tissue product, or a homogenized tissue product) is used as structural (tectonic) support the fascia. In some embodiments, a tissue product described herein (e.g., a flat tissue product sheet, a pulverized tissue product, or a homogenized tissue product) is used as a replacement or supplement for the fascia. In some embodiments, a tissue product described herein (e.g., a flat tissue product sheet, a pulverized tissue product, or a homogenized tissue product) is used to repair a hernia (e.g., to repair the fascia). In some embodiments, a tissue product described herein (e.g., a flat tissue product sheet, a pulverized tissue product, or a homogenized tissue product) is used to repair an inguinal hernia. In some embodiments, a tissue product described herein (e.g., a flat tissue product sheet, a pulverized tissue product, or a homogenized tissue product) is used to repair a femoral hernia. In some embodiments, a tissue product described herein (e.g., a flat tissue product sheet, a pulverized tissue product, or a homogenized tissue product) is used to repair an umbilical hernia. In some embodiments, a tissue product described herein (e.g., a flat tissue product sheet, a pulverized tissue product, or a homogenized tissue product) is used to repair an incisional hernia. In some embodiments, a tissue product described herein (e.g., a flat tissue product sheet, a pulverized tissue product, or a homogenized tissue product) is used to repair a diaphragmatic hernia. In some embodiments, a tissue product described herein (e.g., a flat tissue product sheet, a pulverized tissue product, or a homogenized tissue product) is used to repair a Cooper's hernia, an epigastric hernia, an hiatal hernia, a Littre's hernia, a lumbar hernia, a maydl hernia, an obturator hernia, a pantaloon hernia, a paraesophageal hernia, a paraumbilical hernia, a perineal hernia, a properitoneal hernia, a Richter's hernia, a sliding hernia, a sciatic hernia, a spigelian hernia, a sports hernia, a Velpeau hernia, or a Amyand's hernia.

In some embodiments, a tissue product described herein (e.g., a flat tissue product sheet, a pulverized tissue product, or a homogenized tissue product) is used to repair a spinal disc herniation. In some embodiments, a tissue product described herein (e.g., a flat tissue product sheet, a pulverized tissue product, or a homogenized tissue product) is used as a protective graft over an incision or tear in a spinal disc. In some embodiments, a tissue product described herein (e.g., a flat tissue product sheet, a pulverized tissue product, or a homogenized tissue product) is used as a protective graft over an incision or tear in an annulus fibrosis. In some embodiments, a tissue product described herein (e.g., a flat tissue product sheet, a pulverized tissue product, or a homogenized tissue product) is used as structural (tectonic) support a spinal disc. In some embodiments, a tissue product described herein (e.g., a flat tissue product sheet, a pulverized tissue product, or a homogenized tissue product) is used as structural (tectonic) support an annulus fibrosis. In some embodiments, a tissue product described herein (e.g., a flat tissue product sheet, a pulverized tissue product, or a homogenized tissue product) is used as a replacement or supplement for a spinal disc. In some embodiments, a tissue product described herein (e.g., a flat tissue product sheet, a pulverized tissue product, or a homogenized tissue product) is used as structural (tectonic) support a spinal disc. In some embodiments, a tissue product described herein (e.g., a flat tissue product sheet, a pulverized tissue product, or a homogenized tissue product) is used as a replacement or supplement for an annulus fibrosis.

In some embodiments, a tissue product described herein (e.g., a flat tissue product sheet, a pulverized tissue product, or a homogenized tissue product) is used as a protective graft over an incision in the brain, or in one (or all) of the meninges (i.e., the dura mater, the pia mater, and/or the arachnoid mater). In some embodiments, a tissue product described herein (e.g., a flat tissue product sheet, a pulverized tissue product, or a homogenized tissue product) is used as structural (tectonic) support for one (or all) of the meninges (i.e., the dura mater, the pia mater, and/or the arachnoid mater). In some embodiments, a tissue product described herein (e.g., a flat tissue product sheet, a pulverized tissue product, or a homogenized tissue product) is used as a replacement for one (or all) of the meninges (i.e., the dura mater, the pia mater, and/or the arachnoid mater).

In some embodiments, a tissue product described herein (e.g., a flat tissue product sheet, a pulverized tissue product, or a homogenized tissue product) is used as a protective graft over an incision in a lung or in the pleura. In some embodiments, a tissue product described herein (e.g., a flat tissue product sheet, a pulverized tissue product, or a homogenized tissue product) is used as structural (tectonic) support for the pleura. In some embodiments, a tissue product described herein (e.g., a flat tissue product sheet, a pulverized tissue product, or a homogenized tissue product) is used as a replacement for the pleura.

In some embodiments, a tissue product described herein (e.g., a flat tissue product sheet, a pulverized tissue product, or a homogenized tissue product) is used as a protective graft over an incision in a tympanic membrane. In some embodiments, a tissue product described herein (e.g., a flat tissue product sheet, a pulverized tissue product, or a homogenized tissue product) is used as structural (tectonic) support for a tympanic membrane. In some embodiments, a tissue product described herein (e.g., a flat tissue product sheet, a pulverized tissue product, or a homogenized tissue product) is used as a replacement for a tympanic membrane.

In some embodiments, a tissue product described herein (e.g., a flat tissue product sheet, a pulverized tissue product, or a homogenized tissue product) is used as a protective graft over an incision in the heart or the pericardium. In some embodiments, a tissue product described herein (e.g., a flat tissue product sheet, a pulverized tissue product, or a homogenized tissue product) is used as structural (tectonic) support for the pericardium. In some embodiments, a tissue product described herein (e.g., a flat tissue product sheet, a pulverized tissue product, or a homogenized tissue product) is used as a replacement for the pericardium.

In some embodiments, a tissue product described herein (e.g., a flat tissue product sheet, a pulverized tissue product, or a homogenized tissue product) is used as a protective graft over an incision in the peritoneum. In some embodiments, a tissue product described herein (e.g., a flat tissue product sheet, a pulverized tissue product, or a homogenized tissue product) is used as structural (tectonic) support for the peritoneum. In some embodiments, a tissue product described herein (e.g., a flat tissue product sheet, a pulverized tissue product, or a homogenized tissue product) is used as a replacement for the peritoneum.

### Ophthalmic Uses

Disclosed herein, in certain embodiments, is the use of a tissue product described herein (e.g., a flat tissue product sheet, a pulverized tissue product, or a homogenized tissue product) for repairing, reconstructing, replacing, or supplementing a recipient's damaged, compromised, or missing ocular tissue. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion. In certain instances, the tissue product comprises proteins, glycans, protein-glycan complexes (e.g., a complex of hyaluronic acid and a heavy chain of I+I and PTX3) and enzymes that promote tissue repair.

In some embodiments, a tissue product described herein (e.g., a flat tissue product sheet, a pulverized tissue product, or a homogenized tissue product) comprises chorion or amnion-chorion as a scaffold, and a plurality of cells integrated into the scaffold.

### Treatment of Glaucoma

As used herein, “Glaucoma” means a disorder characterized by the loss of retinal ganglion cells in the optic nerve. In certain instances, glaucoma partially or fully results from an increase in intraocular pressure in the anterior chamber (AC). Intraocular pressure varies depending on the production of liquid aqueous humor by the ciliary processes of the eye and the drainage of the aqueous humor through the trabecular meshwork.

Glaucoma Drainage Devices (GDD) are medical devices that are implanted into an eye to relieve intraoccular pressure by providing an alternative pathway for the aqueous humor to drain. If left uncovered, a GDD tube will erode and leave the eye susceptible to intraocular infection. Thus, the GDD tube needs to be covered. Currently, patches used to cover GDD tubes are made from pericardium, sclera and cornea. These patches are about 400-550 microns thick. The thinness of these patches results in their melting by 25% in 2 years potentially leaving the shunt tube exposed again.

Disclosed herein, is the use of a tissue product described herein (e.g., a flat tissue product sheet, a pulverized tissue product, or a homogenized tissue product) as a patch to cover GDD tubes. In some embodiments, the patch comprises chorion or amnion-chorion. In some embodiments, a tissue product described herein (e.g., a flat tissue product sheet, a pulverized tissue product, or a homogenized tissue product) is 300-600 microns thick. In some embodiments, a tissue product described herein (e.g., a flat tissue product sheet, a pulverized tissue product, or a homogenized tissue product) does not melt by 25% in 2 years.

### Treatment of Ocular Ulcers

Disclosed herein, is the use of a tissue product described herein (e.g., a flat tissue product sheet, a pulverized tissue product, or a homogenized tissue product) as a patch to cover persistent epithelial defects and/or ulcers in eyes.

In some embodiments the base of the ulcer is debrided with surgical sponges and the poorly adherent epithelium adjacent to the edge of the ulcer is removed (e.g., to the section of the eye where the epithelium becomes quite adherent). In some embodiments, a tissue product described herein (e.g., a flat tissue product sheet, a pulverized tissue product, or a homogenized tissue product) is transferred to the recipient eye, with the stromal surface facing the eye and fitted to cover the defect by trimming off the excess edges of a tissue product described herein (e.g., a flat tissue product sheet, a pulverized tissue product, or a homogenized tissue product). In some embodiments, a tissue product described herein (e.g., a flat tissue product sheet, a pulverized tissue product, or a homogenized tissue product) is then secured to the eye by sutures (e.g., interrupted 10-0 nylon sutures or running 10-0 nylon sutures) with the suture knots being buried. In some embodiments, a tissue product described herein (e.g., a flat tissue product sheet, a pulverized tissue product, or a homogenized tissue product) is secured to the eye by use of fibrin glue. In some embodiments, a protective layer is applied over a tissue product described herein (e.g., a flat tissue product sheet, a pulverized tissue product, or a homogenized tissue product) or the entire eye (e.g., a contact lens). In some embodiments, an antibiotic is applied to a tissue product described herein (e.g., a flat tissue product sheet, a pulverized tissue product, or a homogenized tissue product) or the entire eye (e.g., neomycin, polymyxin b sulfate and dexamethasone).

### Conjunctival, Scleral, Lid, and Orbital Rim Surface Reconstruction

Disclosed herein, in certain embodiments, is the use of a tissue product described herein (e.g., a flat tissue product sheet, a pulverized tissue product, or a homogenized tissue product) in conjunctival, scleral, lid, and orbital rim surface reconstruction. In some embodiments, damage to the conjunctival surface results from symblepharon lysis; surgical removal of tumor, lesion, and/or scar tissue; excimer laser photorefractive keratectomy and therapeutic keratectomy; or combinations thereof.

### Cell Transplant

Disclosed herein, in some embodiments, is the use of a tissue product described herein (e.g., a flat tissue product sheet, a pulverized tissue product, or a homogenized tissue product) as a scaffold for transplanting a plurality of cells (e.g., progenitor cells) to a host. In some embodiments, the cells (e.g., progenitor cells) are transplanted to a retina. In some embodiments, at least one hole is formed in a retina. In some embodiments, a retina is partially detached. In some embodiments, the scaffold and cells to be transplanted are placed on the target site.

### Coronary Uses

Disclosed herein, in certain embodiments, is the use of a tissue product described herein (e.g., a flat tissue product sheet, a pulverized tissue product, or a homogenized tissue product) for repairing, reconstructing, replacing, or supplementing a recipient's damaged, compromised, or missing coronary tissue. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion. In certain instances, the tissue product comprises proteins, glycans, protein-glycan complexes (e.g., a complex of hyaluronic acid and a heavy chain of IαI and PTX3) and enzymes that promote tissue repair.

In some embodiments, a tissue product described herein (e.g., a flat tissue product sheet, a pulverized tissue product, or a homogenized tissue product) comprises chorion or amnion-chorion as a scaffold, and a plurality of cells integrated into the scaffold. In some embodiments, the cells are mesothelial cells (e.g., to treat to a wound (e.g., surgical incision) in an internal organ).

### Coronary Artery Bypass

Disclosed herein, is the use of a tissue product described herein (e.g., a flat tissue product sheet, a pulverized tissue product, or a homogenized tissue product) in coronary artery bypass surgery. In some embodiments, tissue product is grafted onto a coronary artery to bypass a section of the artery that is characterized by atherosclerosis. In some embodiments, a tissue product described herein (e.g., a flat tissue product sheet, a pulverized tissue product, or a homogenized tissue product) comprises chorion or amnion-chorion. In some embodiments, a tissue product described herein (e.g., a flat tissue product sheet, a pulverized tissue product, or a homogenized tissue product) described herein is cultured with a plurality of fibroblasts and endothelial cells.

### Heart Valves

In some embodiments, a tissue product described herein (e.g., a flat tissue product sheet, a pulverized tissue product, or a homogenized tissue product) is used as a protective graft over a heart valve. In some embodiments, a tissue product described herein (e.g., a flat tissue product sheet, a pulverized tissue product, or a homogenized tissue product) is used as structural (tectonic) support for a heart valve. In some embodiments, a tissue product described herein (e.g., a flat tissue product sheet, a pulverized tissue product, or a homogenized tissue product) is used as a replacement for a heart valve.

### Veins and Arteries

In some embodiments, a tissue product described herein (e.g., a flat tissue product sheet, a pulverized tissue product, or a homogenized tissue product) is used as a protective covering over a vein or artery. In some embodiments, a tissue product described herein (e.g., a flat tissue product sheet, a pulverized tissue product, or a homogenized tissue product) is used as structural (tectonic) support for a vein or artery. In some embodiments, a tissue product described herein (e.g., a flat tissue product sheet, a pulverized tissue product, or a homogenized tissue product) is used as a replacement for vein or artery.

### Nerve Uses

Disclosed herein, in certain embodiments, is the use of a tissue product described herein (e.g., a flat tissue product sheet, a pulverized tissue product, or a homogenized tissue product) for repairing, reconstructing, replacing, or supplementing a recipient's damaged, compromised, or missing nerve. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion. In certain instances, the tissue product comprises proteins, glycans, protein-glycan complexes (e.g., a complex of hyaluronic acid and a heavy chain of IαI and PTX3) and enzymes that promote tissue repair. For example, the stroma of chorion contains growth factors, anti-angiogenic and anti-inflammatory proteins, as well as natural inhibitors to various proteases. In some embodiments, proteins and enzymes found in the chorion diffuse out of the chorion and into the surrounding tissue.

In some embodiments, a tissue product described herein (e.g., a flat tissue product sheet, a pulverized tissue product, or a homogenized tissue product) is used as a covering over a nerve (e.g., a peripheral nerve). In some embodiments, a tissue product described herein (e.g., a flat tissue product sheet, a pulverized tissue product, or a homogenized tissue product) is used as a covering over a nerve graft, nerve transfer, or a repaired nerve. In some embodiments, a tissue product described herein (e.g., a flat tissue product sheet, a pulverized tissue product, or a homogenized tissue product) is used as a covering over an incision in a nerve (e.g., a peripheral nerve). In some embodiments, a tissue product described herein (e.g., a flat tissue product sheet, a pulverized tissue product, or a homogenized tissue product) is used as structural (tectonic) support for a nerve (e.g., a peripheral nerve). In some embodiments, a tissue product described herein (e.g., a flat tissue product sheet, a pulverized tissue product, or a homogenized tissue product) prevents adhesive in nerve repair.

In some embodiments, a tissue product described herein (e.g., a flat tissue product sheet, a pulverized tissue product, or a homogenized tissue product) is used as a non-constricting encasement for injured nerves. In some embodiments, a tissue product described herein (e.g., a flat tissue product sheet, a pulverized tissue product, or a homogenized tissue product) prevents or minimizes scar formation, encapsulation, chronic compression, tethering of a nerve, and nerve entrapment. In some embodiments, a tissue product described herein (e.g., a flat tissue product sheet, a pulverized tissue product, or a homogenized tissue product) prevents or minimizes neuroma formation. In some embodiments, a tissue product described herein (e.g., a flat tissue product sheet, a pulverized tissue product, or a homogenized tissue product) prevents or minimizes the migration of endogenous growth factors (i.e. Nerve Growth Factor) present during nerve repair.

### Spinal Uses

Disclosed herein, in certain embodiments, is the use of a tissue product described herein (e.g., a flat tissue product sheet, a pulverized tissue product, or a homogenized tissue product) during spinal surgery. In some embodiments, a tissue product described herein (e.g., a flat tissue product sheet, a pulverized tissue product, or a homogenized tissue product) during a laminectomy. In some embodiments, the use is a homologous use. In some embodiments, a tissue product described herein (e.g., a flat tissue product sheet, a pulverized tissue product, or a homogenized tissue product) is minimally manipulated. In some embodiments, a tissue product described herein (e.g., a flat tissue product sheet, a pulverized tissue product, or a homogenized tissue product) does not comprise another article, except for water, crystalloids, or a sterilizing, preserving, or storage agent. In some embodiments, a tissue product described herein (e.g., a flat tissue product sheet, a pulverized tissue product, or a homogenized tissue product) does not have a systemic effect and is not dependent upon the metabolic activity of living cells for its primary function.

In some embodiments, a tissue product described herein (e.g., a flat tissue product sheet, a pulverized tissue product, or a homogenized tissue product) comprises chorion or amnion-chorion.

In certain instances, the tissue product comprises proteins, glycans, protein-glycan complexes (e.g., a complex of hyaluronic acid and a heavy chain of IαI and PTX3) and enzymes that promote tissue repair.

In some embodiments, a tissue product described herein (e.g., a flat tissue product sheet, a pulverized tissue product, or a homogenized tissue product) is used to reduce or prevent epidural fibrosis and/or scar adhesives following spinal surgery (e.g., laminectomy). In some embodiments, a tissue product described herein (e.g., a flat tissue product sheet, a pulverized tissue product, or a homogenized tissue product) is implanted between dura mater and overlying tissue following spinal surgery (e.g., laminectomy). In some embodiments, implanting a tissue product described herein (e.g., a flat tissue product sheet, a pulverized tissue product, or a homogenized tissue product) between dura mater and overlying tissue following spinal surgery (e.g., laminectomy) reduces or prevents migration of fibroblasts to the dura mater and collagen deposition on the dura mater.

In some embodiments, a tissue product described herein (e.g., a flat tissue product sheet, a pulverized tissue product, or a homogenized tissue product) is used to reduce or prevent the development of proliferative scarring following spinal surgery (e.g., laminectomy). In some embodiments, a tissue product described herein (e.g., a flat tissue product sheet, a pulverized tissue product, or a homogenized tissue product) is used to reduce or prevent the development of a postoperative (e.g., postlaminectomy) epidural/peridural/perineural scar. In some embodiments, a tissue product described herein (e.g., a flat tissue product sheet, a pulverized tissue product, or a homogenized tissue product) is used to reduce or prevent the development of proliferative scarring following spinal surgery (e.g., laminectomy). In some embodiments, a tissue product described herein (e.g., a flat tissue product sheet, a pulverized tissue product, or a homogenized tissue product) is used to reduce or prevent the development of a postlaminectomy membrane.

In some embodiments, a tissue product described herein (e.g., a flat tissue product sheet, a pulverized tissue product, or a homogenized tissue product) is used to reduce or prevent the development of extradural compression or dural teethering following spinal surgery (e.g., laminectomy). In some embodiments, a tissue product described herein (e.g., a flat tissue product sheet, a pulverized tissue product, or a homogenized tissue product) is used to reduce or prevent the development of tethered nerve roots following spinal surgery (e.g., laminectomy). In some embodiments, a tissue product described herein (e.g., a flat tissue product sheet, a pulverized tissue product, or a homogenized tissue product) is used to reduce or prevent the development of arachnoiditis following spinal surgery (e.g., laminectomy).

In some embodiments, a tissue product described herein (e.g., a flat tissue product sheet, a pulverized tissue product, or a homogenized tissue product) comprises chorion or amnion-chorion as a scaffold, and a tissue integrated into the scaffold. In some embodiments, the tissue is morcelized bone tissue. In some embodiments, a tissue product described herein (e.g., a flat tissue product sheet, a pulverized tissue product, or a homogenized tissue product) further comprising a tissue integrated into the scaffold (e.g., morcelized bone tissue) is used during a spinal fusion procedure. In some embodiments, a tissue product described herein (e.g., a flat tissue product sheet, a pulverized tissue product, or a homogenized tissue product) further comprising a tissue integrated into the scaffold (e.g., morcelized bone tissue) is implanted between adjacent vertebrae. In some embodiments, implantation of a tissue product described herein (e.g., a flat tissue product sheet, a pulverized tissue product, or a homogenized tissue product) further comprising a tissue integrated into the scaffold (e.g., morcelized bone tissue) is implanted between two adjacent vertebrae promotes fusion of the vertebrae.

In some embodiments, a tissue product described herein (e.g., a flat tissue product sheet, a pulverized tissue product, or a homogenized tissue product) is used as a protective graft over an incision in the dura mater. In some embodiments, a tissue product described herein (e.g., a flat tissue product sheet, a pulverized tissue product, or a homogenized tissue product) is used as structural (tectonic) support for the dura mater. In some embodiments, a tissue product described herein (e.g., a flat tissue product sheet, a pulverized tissue product, or a homogenized tissue product) is used as a replacement for the dura mater.

### Cosmetic Uses

In some embodiments, a tissue product described herein (e.g., a flat tissue product sheet, a pulverized tissue product, or a homogenized tissue product) is used as a dermal filler. In some embodiments, a tissue product described herein (e.g., a flat tissue product sheet, a pulverized tissue product, or a homogenized tissue product) is injected into subdermal facial tissues. In some embodiments, a tissue product described herein (e.g., a flat tissue product sheet, a pulverized tissue product, or a homogenized tissue product) is injected under wrinkles and aging lines of the face (e.g., nasolabial folds, melomental folds, “crow's feet” and forehead wrinkles). In some embodiments, a tissue product described herein (e.g., a flat tissue product sheet, a pulverized tissue product, or a homogenized tissue product) is used for lip augmentation. In some embodiments, a tissue product described herein (e.g., a flat tissue product sheet, a pulverized tissue product, or a homogenized tissue product) is injected into the lips.

### Arthritis

In some embodiments, a tissue product described herein (e.g., a flat tissue product sheet, a pulverized tissue product, or a homogenized tissue product) is used to treat arthritis (e.g., osteoarthritis, rheumatoid arthritis, septic arthritis, ankylosing spondylitis, spondylosis). In some embodiments, a tissue product described herein (e.g., a flat tissue product sheet, a pulverized tissue product, or a homogenized tissue product) is injected into an arthritic joint (e.g., a knee).

### Cell Culture

Disclosed herein, in some embodiments, is the use of a tissue product described herein (e.g., a flat tissue product sheet, a pulverized tissue product, or a homogenized tissue product) as a scaffold for ex vivo expansion of cells (i.e., culturing a plurality of cells). In some embodiments, the cells are embryonic stem cells, mesenchymal stem cells, induced pluripotent stem cells, or any combination thereof. In some embodiments, a tissue product described herein (e.g., a flat tissue product sheet, a pulverized tissue product, or a homogenized tissue product) is used as a scaffold for culturing a plurality of keratocytes. In some embodiments, a tissue product described herein (e.g., a flat tissue product sheet, a pulverized tissue product, or a homogenized tissue product) is used as a scaffold for culturing a plurality of fibroblasts. In some embodiments, a tissue product described herein (e.g., a flat tissue product sheet, a pulverized tissue product, or a homogenized tissue product) is used as a scaffold for culturing a plurality of retinal pigment epithelial (RPE) cells. In some embodiments, a tissue product described herein (e.g., a flat tissue product sheet, a pulverized tissue product, or a homogenized tissue product) is used as a scaffold for culturing a plurality of epithelial cells. In some embodiments, a tissue product described herein (e.g., a flat tissue product sheet, a pulverized tissue product, or a homogenized tissue product) is used as a scaffold for culturing a plurality of epithelial stem cells. In some embodiments, a tissue product described herein (e.g., a flat tissue product sheet, a pulverized tissue product, or a homogenized tissue product) is used as a scaffold for culturing a plurality of limbal stem cells.

In some embodiments, at least one cell is contacted with a tissue product described herein (e.g., a flat tissue product sheet, a pulverized tissue product, or a homogenized tissue product). In some embodiments, the cell is cultured on the tissue product under conditions suitable for growth for a period of time sufficient to produce a plurality of cells.

### EXAMPLES

### Example 1

### Preparation of a Tissue Product Comprising Chorion

Place the isolated chorion into a bowl with prepared PBS and rinse gently. Discard the used rinsing solution into a waste container and repeat the rinsing process until the used rinsing solution appears clear. Transfer the chorion into a sterile bottle and fill with prepared PBS. Allow the chorion to sit for at least 15 minutes. Change the solution in the bottle containing the chorion with clean prepared PBS. Allow the chorion to sit in the clean solution for at least one hour. Gentle swirling of bottle is allowed.

Discard the rinsing solution and pour the chorion into a bowl containing clean prepared PBS. Do not manually clean chorion. Only remove hanging pieces of tissue. Do not use forceps to remove or separate any layers of tissue. Obtain the designated backing

Place the chorion onto a processing tray. Layout the chorion with the side that adheres to the tray towards the tray. Ensure that the chorion is stretched out and that there are no folds on the tissue. Take care not to over stretch the tissue to prevent tears or post cutting shrinking. Proceed to attach the backing to the chorion. Cut an outline of the tissue that is attached to the backing with a scalpel. Place the tissue with the backing into a tray containing clean PBS. Cut the tissue with the backing into the corresponding size using a retractor or ruler and a scalpel. Measure the cut unit and verify that it meets the acceptance criteria for the corresponding catalog size. Check the unit for the presence of debris or tears in tissue or backing Remove any debris if possible, otherwise reject any defective unit. Repeat the cutting and inspection procedure described above until all the tissue is made into product units.

Inspect the product prior to placing in pouch. If applicable reject any defective unit. Place each unit into an inner pouch containing the designated amount of prepared preservation media. Seal each inner pouch using the heat sealer. Place the sealed inner pouch into an outer pouch and heat seal accordingly. Place the designated label on each sealed outer pouch.

### Example 2

### Effective Media and Method for Removing Blood Color using Chorion Sample Tissues

Chorion (CH) from a single donor is previously frozen, and rinsed with PBS to remove blood and clots on the membrane surface. CH is cut into 2.5 cm×2.5 cm pieces, weighed and placed into 150 mm culture dishes containing various solutions (see below) with the sample size (n=3) at 100× volume of the weight of each sample with agitation by a plate shaker (4^{th} speed bar) at room temperature. The samples are visually inspected and pictures are taken at 0, 5, 15, 30 min, 1 hr, 2 hr, 4 hr and 24 hrs.

TABLE 1
Comparison of CH samples in various media by visual observation

| | Visual Observation after 4 hours | | | |
|---|---|---|---|---|
| Media | Color | Texture | Size | Other Observations |
| PBS 1x | Pale pink and | No Change | No Change | Media turns pink |
| (−Agitation) | some red | | | |
| PBS 1x | Pale pink | No Change | No Change | Media turns pink |
| 0.02% NaOCl | Pale yellow | No Change | No Change | Media remains clear |
| | | | | with tinge of yellow |
| 0.05% NaOCl | Pale yellowish | Slightly tougher | Slightly smaller | Media remains clear |
| | brown | | | with tinge of yellow |
| | | | | CH samples turn |
| | | | | yellowish brown |
| | | | | instantaneously |
| 1% Acetic | Yellow Brown | Tougher | Slightly smaller | Media remains clear |
| Acid | | | | with tinge of yellow. |
| | | | | Media releases sour |
| | | | | odor. CH samples turn |
| | | | | yellowish brown |
| | | | | instantaneously |
| 1% HCl | Brown | Tough and | Smaller | Media remains clear |
| | | curled edges | | with tinge of yellow. |
| | | | | CH samples turn brown |
| | | | | instantaneously |
| Acidified ethyl | Opaque white | Tough and | Much smaller | Acidified ethyl acetate |
| acetate for 5 mins | and some brown | bumpy | | media is very pungent |
| followed | | | | and murky white. CH |
| by 1% HCl | | | | samples shrivel and |
| | | | | turn opaque white & |
| | | | | brown. No further |
| | | | | changes observed after |
| | | | | immersion in 1% HCl |

### Conclusion:

Immersion in PBS and 0.02% NaOCl in PBS with agitation most effectively remove blood color while retaining the size and texture of the CH samples compared to other media.

Agitation facilitates blood removal.

Blood retained in CH membranes can also be effectively removed by immersion in PBS and <0.02% NaOCl. Although an optimal concentration of NaOCl (<0.02%) has not been determined, low concentrations of NaOCl can be used as a sequential immersion medium following PBS to remove blood color that cannot be removed by PBS. In addition, the rate of agitation can be increased and media renewed during immersion to avoid saturation.

### Example 3

### To Optimize Blood Color Removal Method with Selected Media using ACMSample Tissues

Amnion-chorion (ACM) is previously frozen, and rinsed with PBS to remove blood and clots on the membrane surface. ACM is cut into 2.5 cm×2.5 cm pieces, and placed into 150 mm culture dishes containing 150 ml of various solutions (see below) with the sample size (n=5) with agitation by a plate shaker (4th speed bar) at room temperature. The samples are visually inspected and pictures are taken at 0, 30 min, 1 hr, 2 hr, 4 hrs.

TABLE 2
Comparison of CH samples in various media by visual observation

| | Visual Observation after 4 hours | | | Other |
|---|---|---|---|---|
| Media | Color | Texture | Size | Observations |
| 1. PBS | Light pink and | No Change | No Change | Media turns pink |
| (−Agitation) | patches of | | | |
| | darker pink | | | |
| 2. PBS | Light pink and | No Change | No Change | Media turns pink |
| | patches of | | | |
| | darker pink | | | |
| 3. 0.005% NaOCl | Light yellowish | No Change | No Change | Media remains |
| | pink with | | | clear with tinge of |
| | patches of | | | yellow |
| | reddish brown | | | |
| 4. 0.01% NaOCl | Yellow Brown | Slightly | Slightly | Media remains |
| | | tougher | smaller | clear with tinge of |
| | | | | yellow. |
| | | | | CH samples turn |
| | | | | yellowish brown |
| | | | | after 5 mins |
| 5. 0.02% NaOCl | Light yellow | Slightly | Slightly | Media remains |
| | with patches | tougher | smaller | clear with tinge of |
| | of brown | | | yellow. CH |
| | | | | samples turn |
| | | | | yellowish brown |
| | | | | after 5 mins |
| 6. PBS for 2 hrs followed by | Light yellowish | No Change | No Change | Media remains |
| 0.005% NaOCl for 2 hrs | pink with | | | clear. |
| | patches of | | | CH samples |
| | light reddish | | | turns from light |
| | brown | | | pink to light |
| | | | | yellowish pink |
| | | | | after 1 hr |

### Conclusion:

Immersion in PBS most effectively remove blood color while retaining the size and texture of the ACM samples compared to other media

All concentrations of NaOCl experimented changes the blood color of samples from red/pink to tinge of yellow but not by removing it from the tissue to the solution. Concentrations of 0.01% and 0.02% NaOCl changes the size and texture of the samples

### Example 4

### To Determine the Effect of Blood Color Removal Methods regarding Protein, HA and HC.HA Complex Contents in ACM Sample Tissues

It is hypothesized that blood color removal methods which involve immersion, agitation and a basic solution media may potentially have damaging effects on the active ingredients of ACM.

It is further hypothesized that agitation and prolonged immersion of ACM samples in NaOCl (a basic solution) may result in significant diffusion and breakdown of proteins, including HA and HC.HA complex. Hence, the content of the active ingredients of the ACM sample tissues processed in various media were measured and compared.

### Experimental Design:

### Sample Preparation

2.5 cm×2.5 cm blood color removed ACM pieces (n=5) are pooled for each of the 6 conditions listed in Aim1b.

The ACM samples were extracted by pulverization and homogenization according to H-089 (samples in conditions 4 & 5 were lost due to broken pestle/mortar). Homogenized samples (n=4) are kept at −80° C. for storage overnight and are used to run the following assays:

i) BCA Protein Assay (Protocol H-013)

Total=(4 samples×3)+(9 standards×2)=30 samples

ii) HC.HA Complex Assay (Protocol H-114)
HABP coated wells from HA Kit are used. HA standards (purified HC.HA (AME)) are diluted at 1:10 and ACM extract samples are diluted at 1:5 with reaction buffer.

Total=(4 samples×3)+(9 standards×2)=30 samples

iii) HA Assay (Kit)
HC.HA wells from ii) are reused. Each well is rinsed 4 times with 360 ul PBS 1×. HA protocol is run starting from Step 7 (HA Kit Assay Procedure) by adding 100 ul HRP-conjugated HABP solution.

### Results:

i) BCA Protein Assay

TABLE 5
Protein Concentrations of ACM Extract Samples after Blood Color
Removal according to BCA

| | Protein Concentration | p-values compared to |
|---|---|---|
| Sample | (ug/ml) | 2) PBS (+Agitation) |
| 1) PBS (−Agitation) | 737 ± 73 | 0.11 |
| 2) PBS (+Agitation) | 585 ± 24 | — |
| 3) 0.005% NaOCl | 711 ± 42 | 0.76 |
| (+Agitation) | | |
| 4) PBS 2 hrs followed by | 586 ± 22 | 0.11 |
| NaOCl 2 hrs (+Agitation) | | |

Samples immersed in 0.005% NaOCl do not show decrease in protein concentration compared to samples in PBS control. However, the protein concentrations for all 4 conditions are significantly lower compared to our previous data for CHE (3652±120 ug/ml). HA and HC.HA concentration readings are not valid due to out of range measurements compared to HA standards.

### Conclusion:

0.005% NaOCl does not decrease the protein concentration of ACM samples compared to control group.

Significantly lower protein concentrations are found in ACM samples after 4 hours of immersion in various media.

### Interpretation:

Agitation and prolonged immersion of ACM samples in NaOCl (a basic solution) can result in significant diffusion and breakdown of proteins.

### Example 5

### Skin Lotion Composition Containing Chorion

A skin lotion is prepared by the following method. 0.25 g methyl hydroxybenzoate and 7.5 g glycerin are dissolved in 75 ml of water at 150° F. 0.7 g sorbitan monolaurate, 0.7 g polysorbate 20, and 1.0 g cetostearyl alcohol are melted at 150° F. and are then compounded into the solution. The mixture is allowed to cool while mixing. When the mixture reaches a temperature below approximately 90° F., 4 ml of chorion pulverized tissue product is added to the mixture. The lotion is packaged into 10 ml aliquots and stored at room temperature.

### Example 6

### Ophthalmic Solution Composition and Treatment of an Eye Disease

An ophthalmic eye drop solution is prepared by mixing 100 mg of chorion pulverized tissue product with 0.9 g of NaCl in 100 mL of purified water and filtered using a 0.2 micron filter. The resulting isotonic solution is then incorporated into ophthalmic delivery units, such as eye drop containers, which are suitable for ophthalmic administration. Two drops of the composition is applied to a burn-damaged eye 4 times per day. By use of this method, the eye returns to normal health.

### Example 7

### Eye Ointment Composition and Treatment of Eye Disease Using Same

A sterile eye ointment composition is prepared by compounding 90 grams white petrolatum, 10 grams liquid petrolatum, and 0.5 grams chorion pulverized tissue product. The mixture is pasteurized and packaged into individual tube containers of 2.0 g each. To treat an eye disease using the composition, an aliquot of approximately 0.1 g is gently applied directly from the tube to the inner edge of the bottom eye lid. The ointment is applied 4 times per day. The patient progress is monitored every other week by an ophthalmologist. By use of this method, the eye disease improves.

### Example 8

### Treatment of Human Eye Disease Using Chorion Tissue Preparation

An individual with burn damage to the eye is identified. A preparation of 1% chorion pulverized tissue product, 0.5% collagen in DMEM is prepared. The individual is treated 4×/day with 2 drops of the composition. By use of this method, the eye damage can be improved as compared to a non-treated burn damaged eye.

### Example 9

### Treatment of Human Skin Disease Using Amnion-Chorion Preparation

An individual with psoriasis is identified. The individual is treated with a 5% preparation of pulverized amnion-chorion tissue product with acceptable excipients. The formulation is dissolved in a lotion composition. The treatment is administered 2 times per day. By use of this method, the psoriasis is alleviated or disappears.

### Example 10

### Rectal Gel Composition Containing Chorion

A rectal gel composition is prepared by combining 100 mg of commercially available HA and with 5 ml sterile chorion, prepared from pulverized chorion tissue product. To this mixture is added 2.5 g of methylcelluose (1500 mPa), 100 mg of methylparapen, 5 g of glycerin and 95 mL of purified water. The resulting gel mixture is then incorporated into rectal delivery units, such as syringes, which are suitable for rectal administration.

### Example 11

### Parenteral Composition

A parenteral composition for intramuscular administration is prepared by mixing 10 mg each of: pulverized chorion tissue product and pulverized amnion-chorion tissue product, with 100 mg of a water-soluble salt of a compound described herein is dissolved in DMSO and then mixed with 10 mL of 0.9% sterile saline. The mixture is incorporated into a dosage unit form suitable for administration by injection.

### Example 12

### Treatment of a Human Tumor by Direct Injection of CH or ACM Preparation

A patient having a subcutaneous tumor of approximately 2 cm in width is identified. 10 grams of reconstituted pulverized chorion tissue product is mixed with 10% PEG 300 in water for 2 hours at 4° C. The mixture is filtered through a 0.20 μm filter, and aliquoted to sterile glass vials which were closed with rubber stoppers and sealed with aluminum caps. The patient is treated by directly injecting the 0.50 ml solution through the skin into the tumor site, dividing the administration volume into 4 separate regions of the tumor mass (approximately 125 μA to each of the four sites). The composition is administered every 48 hours. The tumor size is monitored weekly. By use of this method the tumor size decreases.

Isolated chorion prepared according to Example 1 was used.

The chorion was adhered with its sticky side up (epithelium side down) onto a piece of Nylon Membrane (NM). The chorion/NM was placed in a 60 mm culture dish.

The chorion was frozen by holding the dish on the surface of liquid nitrogen. Caution was taken to ensure that the liquid nitrogen did not overflow into the dish and come in contact with the chorion/NM.

The frozen chorion/NM was lyophilized by placing it in a vacuum chamber of a lyophilization machine for 20 hours.

Once lyophilization was complete, the chorion was detached from the NM by slowly peeling it off. It was then placed it in a pouch for storage and exposed to gamma radiation at 25 kGy.

### Example 14

### Rehydration of Lyophilized Chorion

Lyophilized chorion prepared according to Example 13 was used.

The lyophilized chorion was placed in PBS1× for 30 minutes.

### Example 15

### Use of a Tissue Product During Laminectomy

An individual in need of a laminectomy is identified. A tissue graft made of chorion is prepared.

Laminectomy is performed. Following laminectomy, the tissue graft made of chorion is place over the remaining vertebrae and affixed. The surgical site is closed and sutured.

### Example 16

### Use of a Tissue Product During Spinal Fusion

An individual in need of a spinal fusion is identified.

A composition comprising pulverized chorion tissue product and morcelized bone tissue is prepared. The vertebrae to be fused are exposed. The composition_is injected between adjacent vertebrae. The surgical site is closed and sutured.

### Example 17

### Use of an Amnion-Chorion Product to Repair Nerve Tissue

An individual in need of nerve repair is identified. A tissue graft made of amnion-chorion is prepared.

The nerve to be repaired is exposed. The tissue graft made of chorion is placed over damage to nerve and sutured in place. The surgical site is closed and sutured.

### Example 18

### Use of an Amnion-Chorion Product to Repair a Hernia

An individual in need of spinal disc herniation repair is identified. A tissue graft made of amnion-chorion is prepared.

The herniated spinal disc is exposed. In some embodiments, a tissue product described herein (e.g., a flat tissue product sheet, a pulverized tissue product, or a homogenized tissue product) is used to repair a spinal disc herniation. The tissue graft made of chorion is placed over damage to the annulus fibrosis and sutured in place. The surgical site is closed and sutured.

### Example 19

### Use of a Tissue Product to Repair a Torn Rotator Cuff

An individual in need of rotator cuff repair is identified. A tissue graft made of chorion is prepared.

The torn supraspinatus tendon is exposed. The tendon is sutured together. The tissue graft made of chorion is placed over the suture site and sutured into place. The surgical site is closed and sutured.

### Example 20

### Use of a Tissue Product to Repair Gingival Recession

An individual in need of gingival replacement is identified. A tissue graft made of chorion is prepared.

The tissue graft of chorion is placed over the area of gingival recession. The graft is sutured into place. A protective covering is place over the graft.

### Example 21

### Use of an Amnion-Chorion Product in Coronary Artery Bypass

An individual in need of coronary artery bypass is identified. A tubular tissue graft made of chorion is prepared by culturing it with a plurality of fibroblasts and endothelial cells.

The individual is placed on bypass. The atherosclerotic section of the artery is exposed and removed. The tubular tissue graft made of chorion is sutured to the open ends of the artery such that the artery is rejoined. The surgical site is closed and sutured.

### Example 22

### Use of a Tissue Product in the Treatment of Glaucoma

An individual in need of a Glaucoma Drainage Device is identified. A tissue graft made of chorion is prepared.

The Glaucoma Drainage Device is implanted into an eye. The tissue graft of chorion is placed over the GDD and sutured into place. A protective contact lens is placed over the tissue graft.

### Example 23

### Use of a Tissue Product in the Treatment of a Diabetic Foot Ulcer

An individual with a diabetic foot ulcer is identified. A tissue graft made of chorion is prepared.

The foot ulcer is debrided. The tissue graft is placed over the ulcer. A protective bandage is placed over the tissue graft.

### Example 24

### Use of an Amnion-Chorion Product in the of a Burn

An individual with a 3^{rd} degree burn is identified. A tissue graft made of chorion is prepared.

The burn is debrided. The tissue graft is placed over the burn. A protective bandage is placed over the tissue graft.

### Example 25

### Use of a Tissue Product Following of a Bladder Tumor

An individual with a bladder tumor is identified. A tissue graft made of chorion is prepared.

The bladder is exposed. The tumor removed from the bladder. The tissue graft is placed over excision site. The surgical site is closed and sutured.

### Example 26

### Use of an Amnion-Chorion Product to Supplement the Tympanic Membrane

An individual with a tympanic membrane is identified. A tissue graft made of amnion-chorion is prepared.

The tympanic membrane is visualized. The tissue graft is placed over tear in the tympanic membrane and sutured into place. A protective covering is placed over the tissue graft.

### Example 27

### Biochemical Characterization

Chorion extract (CHE) has lower HA concentration and higher protein concentration than amniotic membrane extract (AME)

| | Extract | | | Purified Extract | | |
|---|---|---|---|---|---|---|
| | Protein | HA | HA:Protein | Protein | HA | HA:Protein |
| | (μg/ml) | (μg/ml) | Ratio | (μg/ml) | (μg/ml) | Ratio |
| AM | 214 ± 36 | 28.6 ± 5.3 | 1:7.5 | undetectable | 41.2 ± 1.8 | — |
| CH | 1826 ± 60 | 0.93 ± 0.03 | 1:1963 | 123 ± 56 | 0.72 ± 0.02 | 1:227 ± 93 |

HA concentration as measured by HA ELISA was significantly lower in CHE compared to AME before and after purification. In contrast, protein concentration as measured by BCA protein assay was significantly higher in CH compared to AM in both the extract and the purified extract.

Purified chorion extract results in higher release of PTX3 and HC1 as compared to purified amniotic membrane extract

Purified extract from amniotic membrane (HC.HA(AM)) and that from chorion (HC.HA(CH)) were treated with or without HAase before western blot analysis with anti-PTX3 or anti-HC1 antibody. Compared with control PTX3, a 90 kD band (dimer) and a smear of higher molecular weight bands were detected in HC.HA(AM) and HC.HA(CH). HAase digestion increased the intensity of these bands, suggesting that PTX3 was bound to HC.HA complex and can be released when HA is removed. However, more PTX3 was released from HC.HA(CH) than that from HC.HA(AM). The similar results were found with anti-HCl antibody.

Purified chorion extract is 25 fold more potent than that from amniotic membrane extract regarding HUVEC proliferation inhibition

The potency of purified extract regarding anti-angiogenic action was compared by proliferation inhibition of HUVEC cells which were seeded simultaneously with HC.HA (CHE) or HC.HA (AME) on 96-well plates for 48 hours and measured by BrdU ELISA. The absorbance values plotted against HC.HA (AME) and HC.HA (CHE) concentration from 0.5-25 μg/ml and 0.025-1 μg/ml respectively fitted logarithmic curves. HC.HA (CHE) was shown to be 25 fold more potent than HC.HA (AME) according to IC50 (3.0 vs. 0.12) based on HA concentration.

### Example 28

### Treatment of Venous Ulcer

An 81 year old female patient developed a venous ulcer on her right medial calf. She received wound care at home but her wound remained slow to heal. She started to wear compression stockings 5 days before treatment with a tissue product comprising chorion. A chorion tissue product was applied on the wound and was overlaid with a wound veil, Vaseline gauze, non-adherent pad and wrapped in. At Week 1, 92.1% of the wound was closed. The tissue product comprising chorion was reapplied with the same external dressings as Week 0. By Week 2, the wound was completely healed.

### Example 29

### Treatment of Venous Ulcer

A 43 year old male patient developed a venous ulcer on his left lateral ankle He has a history of slow healing wounds of the lower legs. The wound was treated with sharp debridement, Santyl® collagenous ointment, Hydrofera Blue® antimicrobial dressing, Tegaderm® sterile film dressing and Actiocoat® silver dressing and showed slight improvement but with no evidence of new granulation tissue.

Following sharp debridement, an a tissue product comprising chorion was applied on the wound, secured with Steristrips, and was overlaid with a wound veil, silver dressing, Vaseline gauze and a multilayer Profore wrap compression therapy.

The wound was evaluated and treated weekly except between Week 9 and Week 12. The tissue product comprising chorion was reapplied on Week 1 and Week 7.5 with external dressings similar to that of Week 0. On weeks when the tissue product comprising chorion was not applied during clinical treatment, the wound was debrided and dressed with the external dressings similar to that of Week 0. At Week 2, the wound had achieved 93.7% closure. The wound was completely healed by Week 12.

### Example 30

### Treatment of Venous Ulcer

An 82 year old female patient developed a venous ulcer on her right lateral ankle For 1 month, her wound was treated with sharp debridement, Santyl® collagenous ointment, Hydrofera Blue® antibacterial dressing, Neosporin® antibiotic dressing, Aquacel® antibacterial dressing, Prisma® antimicrobial dressing and compression therapy but remained slow to heal.

A week before the application of a tissue product comprising chorion, she underwent a radiofrequency ablation procedure of the right saphenous vein. A tissue product comprising chorion was then applied on the wound following sharp debridement, and was overlaid with a wound veil, silver dressing, Vaseline gauze and a multilayer compression wrap. Her wound was evaluated and treated weekly. The tissue product comprising chorion was reapplied on Week 1 with external dressings similar to that of Week 0. After Week 2, the wound was treated with Acticoat® silver dressing and compression therapy. The wound was completely healed by Week 5.

### Example 31

### Treatment of Arterial Insufficiency Ulcer

A 72 year old male diabetic patient with arterial insufficiency developed an ulcer on his right plantar foot. He was treated with sharp debridement, Acticoat, Aquasol® silver dressing or Hydrofera Blue® antibacterial dressing but remained slow to heal.

Following sharp debridement, a tissue product comprising chorion was applied on the wound and was overlaid with a wound veil, Vaseline gauze, Telfa® non adhesive pad and Kerlix bandage. The wound was evaluated and clinically treated weekly except on Week 5. The tissue product comprising chorion was reapplied on Week 1, 2, and 3 with the same external dressing as Week 0. By Week 2, the wound had achieved 89.5% closure. After Week 3, the wound has been treated with Bactroban® antibacterial ointment, Vaseline gauze and Kerlix® bandage. The wound was completely healed by Week 6.

### Example 32

### Treatment of Arterial Insufficiency Ulcer

A 72 year old male diabetic patient with arterial insufficiency developed a recurring ulcer on his right lateral foot. The ulcer was treated with sharp debridement, Acticoat®, Aquasol® silver dressing or Hydrofera Blue® antibacterial dressing but remained non-healing.

Following sharp debridement, a tissue product comprising chorion was applied on the wound, secured with Steristrips, and was overlaid with a wound veil, Vaseline gauze, Telfa® non adhesive pad and Kerlix® bandage. The wound was evaluated and treated weekly except on Week 5. The tissue product comprising chorion was reapplied on Week 1, 2, 3, 6, 7, and 8 with the same external dressing as Week 0. On Week 4, when the tissue product comprising chorion was not applied, the wound was treated with Bactroban® antibiotic ointment, Vaseline gauze and Kerlix dressing. By Week 8, the wound had achieved 54.8% closure.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A tissue product, comprising: isolated chorion that is substantially free of cells with metabolic activity, wherein the natural biological activity of the isolated chorion is substantially maintained.

2. The tissue product of claim 1, wherein the natural structural integrity of the isolated chorion is substantially maintained.

3. (canceled)

4. The tissue product of claim 1, wherein the tissue product is cryopreserved, lyophilized, terminally sterilized, or any combination thereof.

5. The tissue product of claim 1, wherein the tissue product is an extract of chorion.

6. The tissue product of claim 1, wherein the tissue product is a substantially-flattened sheet.

7. The tissue product of claim 1, wherein the tissue product is a pulverized powder or a homogenate.

8. (canceled)

9. (canceled)

10. (canceled)

11. (canceled)

12. (canceled)

13. (canceled)

14. A tissue product, comprising: isolated amnion-chorion that is substantially free of cells with metabolic activity, wherein the natural biological activity of the isolated amnion-chorion is substantially maintained.

15. The tissue product of claim 14, wherein the natural structural integrity of the isolated amnion-chorion is substantially maintained.

16. (canceled)

17. The tissue product of claim 14, wherein the tissue product is cryopreserved, lyophilized, terminally sterilized, any combination thereof.

18. (canceled)

19. The tissue product of claim 14, wherein the tissue product is a substantially-flattened sheet.

20. The tissue product of claim 14, wherein the tissue product is a pulverized powder or a homogenate.

21. (canceled)

22. (canceled)

23. (canceled)

24. (canceled)

25. (canceled)

26. (canceled)

27. (canceled)

28. (canceled)

29. (canceled)

30. (canceled)

31. The tissue product of claim 1, wherein the chorion is isolated from fresh, frozen or previously frozen placenta.

32. The tissue product of claim 14, wherein the amnion-chorion is isolated from fresh, frozen or previously frozen placenta.

33. A composition comprising, chorion that is isolated from placenta and is substantially free of cells with metabolic activity, wherein the natural biological activity of the chorion is substantially maintained.

34. The composition of claim 33, wherein the chorion is isolated from fresh, frozen or previously frozen placenta.

35. The composition of claim 33, further comprising a pharmaceutically-acceptable excipient.

36. The composition of claim 33, in the form of a cream, lotion, ointment, paste, gel, film, or stick.

37. A composition comprising, amnion-chorion that is isolated from placenta and is substantially free of cells with metabolic activity, wherein the natural biological activity of the amnion-chorion is substantially maintained.

38. The composition of claim 37, wherein the amnion-chorion is isolated from fresh, frozen or previously frozen placenta.

39. The composition of claim 37, further comprising a pharmaceutically-acceptable excipient.

40. The composition of claim 37, in the form of a cream, lotion, ointment, paste, gel, film, or stick.

41. A wound dressing or patch comprising, chorion that is isolated from placenta and is substantially free of cells with metabolic activity, wherein the natural biological activity of the chorion is substantially maintained.

42. The wound dressing of claim 41, wherein the chorion is isolated from fresh, frozen or previously frozen placenta.

43. A wound dressing or patch comprising, amnion-chorion that is isolated from placenta and is substantially free of cells with metabolic activity, wherein the natural biological activity of the amnion-chorion is substantially maintained.

44. The wound dressing of claim 41, wherein the amnion-chorion is isolated from fresh, frozen or previously frozen placenta.
